(19) United States
(12) Patent Application Publication
(10) Pub. No.: US 2018/0155429 A1
(43) Pub. Date: Jun. 7, 2018

(54) TREATMENT OF PD-L1 POSITIVE LUNG CANCER USING AN ANTI-PD-1 ANTIBODY

(71) Applicant: Bristol-Myers Squibb Company, Princeton, NJ (US)

(72) Inventor: Friedrich FINCKENSTEIN, Princeton, NJ (US)

(73) Assignee: Bristol-Myers Squibb Company, Princeton, NJ (US)

(21) Appl. No.: 15/577,673

(22) PCT Filed: May 27, 2016
(86) PCT No.: PCT/US2016/034869

§ 371 Date: Nov. 28, 2017

Related U.S. Application Data
(60) Provisional application No. 62/167,674, filed on May 28, 2015.

(51) Int. Cl. C07K 16/28; A61P 35/00; C07K 16/30; A61K 9/00; A61K 39/395; A61K 45/06; G01N 33/574

(52) U.S. Cl. CPC C07K 16/2827; A61P 35/00; C07K 16/3023; A61K 9/0019; A61K 39/39558; A61K 2039/545; A61K 39/3955; G01N 33/57423; C07K 2317/76; C07K 2317/24; C07K 2317/21; A61K 45/06

(57) ABSTRACT

This disclosure provides a method for treating a subject afflicted with a PD-L1 positive non-squamous non-small cell lung cancer (NSCLC). The method comprises administering to the subject a therapeutically effective amount of an anti-PD-1 anti-body or anti-PD-L1 antibody. In some embodiments, the non-squamous NSCLC tumor has a PD-L1 expression of at least about 1%. In some embodiments, the subject is additionally administered another anti-cancer agent.

Throughout this application, various publications are referenced in parentheses by author name and date, or by Patent No. or Patent Publication No. Full citations for these publications can be found at the end of the specification immediately preceding the claims. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

### FIELD OF THE INVENTION

This invention relates to improving therapeutic efficacy of an anti-PD-1 antibody or an anti-PD-L1 antibody therapy by identifying a subject who responds better to the therapy, in particular, a subject who has a tumor expressing PD-L1. For example, the invention includes methods for treating PD-L1 positive non-squamous, non-small cell lung cancer (NSCLC) in a subject comprising administering to the subject an anti-PD-1 antibody or an anti-PD-L1 antibody.

### BACKGROUND OF THE INVENTION

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of an antibody (Ab), ipilimumab (YERVOY®), that binds to and inhibits CTLA-4 for the treatment of patients with advanced melanoma (Hodi et al. (2010) N Engl J Med 363:711-23) and the development of antibodies such as nivolumab and pembrolizumab (formerly lambrolizumab; USAN Council Statement (2013) Pembrolizumab: Statement on a nonproprietary name adopted by the USAN Council (ZZ-165), Nov. 27, 2013) that bind specifically to the Programmed Death-1 (PD-1) receptor and block the inhibitory PD-1/PD-1 ligand pathway (Topalian et al. (2012a) N Engl J Med 366:2443-54; Topalian et al. (2012b) Curr Opin Immunol 24:207-12; Topalian et al. (2014) J Clin Oncol 32(10):1020-30; Hamid et al. (2013) N Engl J Med 369:134-144; Hamid and Carvajal (2013) Expert Opin Biol Ther 13(6):847-61; McDermott and Atkins (2013) Cancer Med 2(5):662-73).

PD-1 is a key immune checkpoint receptor expressed by activated T and B cells and mediates immunosuppression. Nivolumab (formerly designated 5C4, BMS-936558, MDX-1106, or ONO-4538) is a fully human IgG4 (S228P) PD-1 immune checkpoint inhibitor Ab that selectively prevents interaction with PD-1 ligands (PD-L1 and PD-L2), thereby blocking the down-regulation of antitumor T-cell functions (U.S. Pat. No. 8,008,449; Wang et al. In vitro characterization of the anti-PD-1 antibody nivolumab, BMS-936558, and in vivo toxicology in non-human primates, Cancer Imm Res, 2(9):846-56 (2014)). Nivolumab has been approved for the treatment of patients with unresectable or metastatic melanoma and disease progression following ipilimumab and, if BRAF V600 mutation positive, a BRAF inhibitor and for the treatment of squamous non-small cell lung cancer.

### SUMMARY OF THE INVENTION

The present disclosure relates to a method for treating a subject afflicted with a tumor derived from a non-squamous, non-small cell lung cancer (NSCLC) comprising administering to the subject a therapeutically effective amount of: an anti-PD-1 antibody or an antigen-binding portion thereof or an anti-PD-L1 antibody or antigen-binding portion thereof, wherein the tumor has a PD-L1 expression of at least about 1%. In certain embodiments, the disclosure relates to a method of treating a subject afflicted with a tumor derived from a non-squamous NSCLC comprising: (i) measuring a PD-L1 expression on the tumor, wherein the tumor has a PD-L1 expression of at least about 1% and (ii) administering to the subject a therapeutically effective amount of an anti-PD-1 antibody or antigen-binding portion thereof or an anti-PD-L1 antibody or antigen-binding portion thereof. In other embodiments, the disclosure relates to a method for identifying a subject afflicted with a tumor derived from a non-squamous NSCLC who is suitable for an anti-PD-1 antibody or anti-PD-L1 antibody therapy comprising measuring a PD-L1 expression on the tumor, wherein the tumor has a PD-L1 expression of at least about 1% and wherein the subject is administered an anti-PD-1 antibody or antigen-binding portion thereof or an anti-PD-L1 antibody or antigen-binding portion thereof. In one embodiment, the disclosure relates to a method for identifying a subject afflicted with a tumor derived from a non-squamous NSCLC who is suitable for an anti-PD-1 antibody or anti-PD-L1 antibody therapy comprising: (i) measuring a PD-L1 expression on the tumor, wherein the tumor has a PD-L1 expression of at least about 1% and (ii) administering to the subject a therapeutically effective amount of an anti-PD-1 antibody or antigen-binding portion thereof or an anti-PD-L1 antibody or antigen-binding portion thereof.

In certain embodiments, the PD-L1 expression of the tumor is at least about 5%. In other embodiments, the PD-L1 expression level of the tumor is at least about 10%.

In some embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the anti-PD-1 antibody or antigen-binding portion thereof comprises a heavy chain constant region which is of a human IgG1 or IgG4 isotype. In one embodiment, the anti-PD-1 antibody is nivolumab. In another embodiment, the anti-PD-1 antibody is pembrolizumab.

In some embodiments, the anti-PD-L1 antibody or antigen-binding fragment thereof competes for binding with BMS-936559, MPDL3280A, MEDI4736 or MSB0010718C for binding to human PD-L1. In certain embodiments, the anti-PD-L1 antibody or antigen-binding portion thereof is a chimeric, humanized or human monoclonal antibody or a portion thereof. In some embodiments, the anti-PD-L1 antibody or antigen-binding portion thereof comprises a heavy chain constant region which is of a human IgG1 or IgG4 isotype. In one embodiment, the anti-PD-L1 antibody is BMS-936559. In another embodiment, the anti-PD-L1 antibody is MPDL3280A. In a further embodiment, the anti-PD-L1 antibody is MEDI4736. In another embodiment, the anti-PD-L1 antibody is MSB0010718C.

In some embodiments, the anti-PD-1 antibody or antigen-binding portion thereof or the anti-PD-L1 antibody or antigen-binding portion thereof is administered at a dose ranging from at least about 0.1 to at least about 10.0 mg/kg body weight once every about 2, about 3 or about 4 weeks. In specific embodiments, the anti-PD-1 antibody or antigen-binding portion thereof or the anti-PD-L1 antibody or antigen-binding portion thereof is administered at a dose of at least about 3 mg/kg body weight once about every 2 weeks.

In certain embodiments, the anti-PD-1 antibody or antigen-binding portion or the anti-PD-L1 antibody or antigen-binding portion thereof is administered for as long as clinical benefit is observed or until unmanageable toxicity or disease progression occurs.

In one embodiment, the tumor of the subject has a PD-L1 expression level that is at least about 5%. In another embodiment, the tumor of the subject has a PD-L1 expression level that is at least about 10%.

In certain embodiments, the subject has had prior treatment with platinum-based doublet chemotherapy (PT-DC).

In some embodiments, the subject exhibits an overall survival of at least about 10 months, at least about 11 months, at least about 12 months, at least about 13 months, at least about 14 months at least about 15 months, at least about 16 months, at least about 17 months, at least about 18 months, at least about 19 months, at least about 20 months, at least about 21 months, at least about 22 months, at least about 23 months, at least about 2 years, at least about 3 years, at least about 4 years, or at least about 5 years after the administration. In one embodiment, the tumor has a PD-L1 expression of at least about 1% and the subject exhibits the overall survival of at least about 17 months. In another embodiment, the tumor has a PD-L1 expression of at least about 5% and the subject exhibits the overall survival of at least about 18 months. In yet another embodiment, the tumor has a PD-L1 expression of at least about 10% and the subject exhibits the overall survival of at least about 19 months.

In other embodiments, the subject has an overall response rate of at least about 30%, 35%, 36%, 37%, 39%, 40%, 45%, or 50% after the administration compared to the response rate after administration of Docetaxel. In one embodiment, the tumor has a PD-L1 expression of at least about 1% and the subject exhibits an overall response rate of at least about 31%. In another embodiment, the tumor has a PD-L1 expression of at least about 5% and the subject exhibits an overall response rate of at least about 35%. In yet another embodiment, the tumor has a PD-L1 expression level of at least about 10% and the subject exhibits an overall response rate of at least about 37%. In other embodiments, the subject exhibits a median duration of response of at least about 16 months.

In certain embodiments, the anti-PD-1 antibody or portion thereof or the anti-PD-L1 antibody or portion thereof is formulated for intravenous administration. In some embodiments, the anti-PD-1 antibody or antigen-binding portion thereof or the anti-PD-L1 antibody or antigen-binding portion thereof is administered at a subtherapeutic dose.

In other embodiments, the method further comprises administering one or more additional anti-cancer agents. In some embodiments, the anti-cancer agent is selected from the group consisting of an antibody or antigen-binding portion thereof that binds specifically to a CTLA-4 and inhibits CTLA-4 activity, a chemotherapy, a platinum-based doublet chemotherapy, a tyrosine kinase inhibitor, or an anti-VEGF inhibitor. In certain embodiments, the anti-cancer agent is an antibody or antigen-binding portion thereof that binds specifically to a CTLA-4 and inhibits CTLA-4 activity.

In some embodiments, the disclosure is directed to a kit for treating a subject afflicted with a tumor derived from a non-squamous NSCLC, the kit comprising: (a) an anti-PD-1 antibody or antigen-binding portion thereof or an anti-PD-L1 antibody or antigen-binding portion thereof; and (b) instructions for measuring a PD-L1 expression of the tumor and administering the anti-PD-1 antibody or antigen-binding portion thereof or an anti-PD-L1 antibody or antigen-binding portion thereof to the subject.

Other features and advantages of the instant invention will be apparent from the following detailed description and examples which should not be construed as limiting. The contents of all cited references, including scientific articles, newspaper reports, GenBank entries, patents and patent applications cited throughout this application are expressly incorporated herein by reference.

### BRIEF DESCRIPTION OF THE DRAWINGS

FIG. 1 shows an overview of the nivolumab(“nivo”)/docetaxel(“doc”) clinical trial. FIG. 1A shows the trial flow chart. FIG. 1B shows the study design.

FIG. 2 shows the overall survival (“OS”) of patients receiving nivolumab and patients receiving docetaxel. The 1-year OS rate of patients receiving nivolumab was 51%, while the 1-year OS rate of patients receiving docetaxel was 39%.

FIG. 3 shows the treatment effect on the OS in predefined subgroups. The hazard ratios are also indicated.

FIG. 4 shows the progression-free survival (PFS) of patients receiving nivolumab and patients receiving docetaxel. The 1-year PFS rate of patients receiving nivolumab was 19%, while the 1-year PFS rate of patients receiving docetaxel was 8.1%.

FIG. 5A shows the overall survival of patients receiving nivolumab and patients receiving docetaxel by PD-L1 expression. FIG. 5B shows the overall survival of patients receiving nivolumab and patients receiving docetaxel by PD-L1 expression in comparison to patients who are PD-L1 negative. The OS for patients with ≥1%, 5%, and 10% PD-L1 expression is shown.

FIG. 6 shows the overall survival and progression-free survival hazard ratios by baseline PD-L1 expression in patients receiving nivolumab and patients receiving docetaxel.

FIG. 7 shows the response characteristics of confirmed responders. The time to first response, the time on treatment, the time off treatment, and ongoing responses are indicated.

FIG. 8 shows the response characteristics of confirmed responders by PD-L1 expression. The time to first response, the time on treatment, the time off treatment, and ongoing response are indicated.

FIG. 9 shows the PFS by PD-L1 expression. The PFS for patients with ≥1%, 5%, and 10% PD-L1 expression is shown.

### DETAILED DESCRIPTION OF THE INVENTION

The present invention relates to the superior effects of an anti-PD-1 antibody or anti-PD-L1 antibody on treating a PD-L1 positive non squamous NSCLC compared to a standard of care therapy (i.e., docetaxel) or compared to the effects on a PD-L1 negative non squamous NSCLC. The present methods for treating a non-squamous NSCLC patient who has a PD-L1 positive tumor comprise administering to the patient an anti-PD-1 antibody or an anti-PD-L1 antibody

### Terms

In order that the present disclosure can be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Routes of administration for the anti-PD-1 antibody include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. The TKI is typically administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 can, however, have cross-reactivity to other antigens, such as PD-1 molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody

A “chimeric antibody” refers to an antibody in which the variable regions are derived from one species and the constant regions are derived from another species, such as an antibody in which the variable regions are derived from a mouse antibody and the constant regions are derived from a human antibody

An “anti-antigen” antibody refers to an antibody that binds specifically to the antigen. For example, an anti-PD-1 antibody binds specifically to PD-1 and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream. In some embodiments, the cancer is lung cancer. In certain embodiments, the cancer is non-small cell lung cancer (NSCLC). In some embodiments, the cancer is non-squamous NSCLC.

“Cytotoxic T-Lymphocyte Antigen-4 (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“PD-L1 positive” as used herein can be interchangeably used with “PD-L1 expression of at least about 1%.” In one embodiment, the PD-L1 expression can be used by any methods known in the art. In another embodiment, the PD-L1 expression is measured by an automated IHC. PD-L1 positive tumor can thus have at least about 1%, at least about 2%, at least about 5%, at least about 10%, or at least about 20% of tumor cells expressing PD-L1 as measured by an automated IHC. In certain embodiments, “PD-L1 positive” means that there are at least 100 cells that express PD-L1 on the surface of the cells.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “subtherapeutic dose” means a dose of a therapeutic compound (e.g., an antibody) that is lower than the usual or typical dose of the therapeutic compound when administered alone for the treatment of a hyperproliferative disease (e.g., cancer).

By way of example, an “anti-cancer agent” promotes cancer regression in a subject or prevents further tumor growth. In certain embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in certain embodiments, relative to patients treated with a standard-of-care therapy. In other embodiments of the invention, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In certain embodiments, the prophylactically effective amount prevents the development or recurrence of the cancer entirely. “Inhibiting” the development or recurrence of a cancer means either lessening the likelihood of the cancer”s development or recurrence, or preventing the development or recurrence of the cancer entirely.

The term “weight based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

The use of the term “flat dose” with regard to the methods and dosages of the invention means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of an antibody (e.g., 240 mg of an anti-PD1 antibody).

The use of the term “fixed dose” with regard to a method of the invention means that two or more different antibodies in a single composition (e.g., anti-PD-1 antibody and anti-CTLA-4 antibody) are present in the composition in particular (fixed) ratios with each other. In some embodiments, the fixed dose is based on the weight (e.g., mg) of the antibodies. In certain embodiments, the fixed dose is based on the concentration (e.g., mg/ml) of the antibodies. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-PD-1 antibody) to mg second antibody (e.g., anti-CTLA-4 antibody). For example, the 3:1 ratio of an anti-PD-1 antibody and an anti-CTLA-4 antibody can mean that a vial can contain about 240 mg of the anti-PD-1 antibody and 80 mg of the anti-CTLA-4 antibody or about 3 mg/ml of the anti-PD-1 antibody and 1 mg/ml of the anti-CTLA-4 antibody.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

Methods of the Invention

This disclosure provides a method of treating a subject afflicted with a PD-L1 positive tumor derived from a non-squamous NSCLC, wherein the method comprises administering to the subject a therapeutically effective amount of an antibody or an antigen-binding portion thereof that specifically binds to and a PD-1 receptor and inhibits PD-1 activity (“anti-PD-1 antibody”) or an antibody or an antigen-binding portion thereof that specifically binds to and a PD-L1 receptor and inhibits PD-L1 activity (“anti-PD-L1 antibody”). The invention shows that an inhibition of interaction between PD-1 and PD-L1 has a superior effect in treating a non-squamous NSCLC; that is, a subject having a non-squamous NSCLC tumor expressing PD-L1 responds better to an anti-PD-1 antibody therapy or an anti-PD-L1 antibody therapy than to a standard of care therapy. In addition, an anti-PD-1 antibody or an anti-PD-L1 antibody has a therapeutically superior effect on treating PD-L1 positive non squamous NSCLC compared to PD-L1 negative non-squamous NSCLC. Therefore, in one embodiment, the invention provides a method of identifying a non-squamous NSCLC subject who responds well to an anti-PD-1 antibody therapy or an anti-PD-L1 antibody therapy comprising measuring the PD-L1 expression on the tumor. In another embodiment, the invention includes a method of identifying a non-squamous NSCLC subject who responds well to an anti-PD-1 antibody therapy or an anti-PD-L1 antibody therapy comprising measuring the PD-L1 expression on the tumor and administering to the subject an anti-PD-1 antibody or antigen-binding portion thereof or an anti-PD-L1 antibody or antigen-binding portion thereof.

The invention also includes methods of determining or identifying a subject afflicted with a non-squamous NSCLC who is suitable for an anti-PD-1 antibody or anti-PD-L1 antibody therapy comprising measuring a PD-L1 expression level on the tumor, wherein the tumor has a PD-L1 expression level of at least about 1% and wherein the subject is administered an anti-PD-1 antibody or antigen-binding portion thereof or an anti-PD-L1 antibody or antigen-binding portion thereof. In other embodiments, the methods for determining or identifying a subject afflicted with a non-squamous NSCLC who is suitable for an anti-PD-1 antibody or anti-PD-L1 antibody therapy comprises: (i) measuring a PD-L1 expression level on the tumor, wherein the tumor has a PD-L1 expression level of at least about 1% and (ii) administering to the subject a therapeutically effective amount of an anti-PD-1 antibody or antigen-binding portion thereof or an anti-PD-L1 antibody or antigen-binding portion thereof.

In certain embodiments, the invention is directed to a method for treating a subject afflicted with a tumor derived from non-squamous NSCLC comprising administering to the subject a therapeutically effective amount of: an anti-PD-1 antibody or anti-PD-L1 antibody, wherein the PD-L1 expression level on the tumor is at least about 1%. In other embodiments, the invention includes a method of treating a subject afflicted with a tumor derived from a non-squamous NSCLC comprising: (i) measuring a PD-L1 expression level on the tumor, wherein the tumor has a PD-L1 expression level of at least 1% and (ii) administering to the subject a therapeutically effective amount of an anti-PD-1 antibody or antigen-binding portion thereof or an anti-PD-L1 antibody or antigen-binding portion thereof.

The PD-L1 status of a tumor in a subject can be measured prior to administering any composition or utilizing any method disclosed herein. In one embodiment, the PD-L1 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20% or greater than at least about 20%. In another embodiment, the PD-L1 status of a tumor is at least about 1%. In other embodiments, the PD-L1 status of the subject is at least about 5%. In a certain embodiment, the PD-L1 status of a tumor is at least about 10%.

In certain embodiments, the therapy of the present invention (e.g., administration of an anti-PD-1 antibody or an anti-PD-L1 antibody and, optionally, another anti-cancer agent) effectively increases the duration of survival of the subject. In some embodiments, the anti-PD-1 antibody therapy or anti-PD-L1 antibody therapy of the present invention increases the duration of survival of the subject in comparison to standard-of-care therapies. In some embodiments, the standard-of-care therapy that the therapy of the present invention is compared to is docetaxel. After the administration of an anti-PD-1 antibody or anti-PD-L1 antibody therapy, the subject having a PD-L1 positive non-squamous NSCLC tumor can exhibit an overall survival of at least about 10 months, at least about 11 months, at least about 12 months, at least about 13 months, at least about 14 months at least about 15 months, at least about 16 months, at least about 17 months, at least about 18 months, at least about 19 months, at least about 20 months, at least about 21 months, at least about 22 months, at least about 23 months, at least about 2 years, at least about 3 years, at least about 4 years, or at least about 5 years after the administration. In a particular embodiment, a subject has a PD-L1 expression level that is at least about 1% and exhibits the overall survival of at least about 17 months. In some embodiments, a subject has a PD-L1 expression level that is at least about 5% and exhibits the overall survival of at least about 18 months. In other embodiments, a subject has a PD-L1 expression level that is at least about 10% and exhibits the overall survival of at least about 19 months.

In other embodiments, the duration of survival or the overall survival of the subject is increased by at least about 1 month, at least about 2 months, at least about 3 months, at least about 4 months, at least about 6 months, or at least about 1 year when compared to another subject treated with only a standard-of-care therapy (e.g., docetaxel). In some embodiments, the overall survival is increased by at least about 4 months, at least about 6 months, at least about 7 months, at least about 8 months, at least about 9 months, at least about 10 months, at least about 11 months, at least about 1 year, at least about 18 months or at least about 2 years when the tumor is PD-L1 positive when compared to another subject treated with only a standard-of-care therapy (e.g., docetaxel). For example, the duration of survival or the overall survival of the subject is increased by at least about 5%, at least about 10%, at least about 15%, at least about 20%, at least about 25%, at least about 30%, at least about 40%, at least about 50% or at least about 75% when compared to another subject treated with only a standard-of-care therapy (e.g., docetaxel).

In certain embodiments, the therapy of the present invention effectively increases the duration of progression free survival of the subject. For example, the progression free survival of the subject is increased by at least about 2 weeks, at least about 1 month, at least about 2 months, at least about 3 months, at least about 4 months, at least about 6 months, or at least about 1 year when compared to another subject treated with only standard-of-care therapy (e.g., docetaxel). For example, the progression free survival of the subject is increased by at least about 2 weeks, at least about 1 month, at least about 2 months, at least about 3 months, at least about 4 months, at least about 6 months, or at least about 1 year when the subject is PD-L1 positive when compared to another subject treated with only standard-of-care therapy (e.g., docetaxel).

In certain embodiments, after the administration of an anti-PD-1 antibody or anti-PD-L1 antibody therapy, the subject exhibits an overall response rate of at least about 30%, 35%, 36%, 37%, 39%, 40%, 45%, or 50% compared to the response rate after administration of a standard-of-care therapy (e.g., Docetaxel). In one embodiment, the tumor has a PD-L1 expression level that is at least about 1% and exhibits an overall response rate of at least about 31%. In another embodiment, the tumor has a PD-L1 expression level that is at least about 5% and exhibits an overall response rate of at least about 35%. In other embodiments, the tumor has a PD-L1 expression level that is at least about 10% and exhibits an overall response rate of at least about 37%. In some embodiments, the therapy of the present invention effectively increases the response rate in a group of subjects. For example, the response rate in a group of subjects is increased by at least about 50%, at least about 25%, at least about 10%, at least about 9%, at least about 8%, at least about 7%, at least about 6%, at least about 5%, at least about 4%, at least about 3%, at least about 2%, or at least about 1% when compared to another group of subjects treated with only standard-of-care therapy (e.g., docetaxel). In certain embodiments, the response rate is increased by at least about 2% when compared to another group of subjects treated with only one therapy. In certain embodiments, the median duration of response is increased by at least about 1 month, at least about 2 months, at least about 3 months, at least about 4 months, at least about 6 months or at least about 1 year when the subject is PD-L1 positive when compared to another subject treated with only a standard-of-care therapy (e.g., docetaxel).

In other embodiments, the subject is a human patient. In certain embodiments, the subject is a chemotherapy-naïve patient (e.g., a patient who has not previously received any chemotherapy). In other embodiments, the subject has received another cancer therapy (e.g., a chemotherapy), but is resistant or refractory to such another cancer therapy. In certain specific embodiments, the subject has cancer cells expressing mutated forms of the EGFR or KRAS gene. In certain embodiments, the PD-L1+ expression level is at least about 1%, at least about 2%, at least about 3%, at least about 4%, at least about 5%, at least about 6%, at least about 7%, at least about 8%, at least about 9%, at least about 10%, at least about 11%, at least about 11%, at least about 12%, at least about 13%, at least about 14%, at least about 15% or at least about 20%. In some embodiments, the subject has non-squamous NSCLC and has a PD-L1 expression level of at least about 1%, at least about 5%, or at least about 10%. In one embodiment, the subject has had a prior systematic treatment. In certain embodiments, the subject has had two or more prior systematic treatments.

In order to assess the PD-L1 expression, in one embodiment, a test tissue sample can be obtained from the patient who is in need of the therapy. In another embodiment, the assessment of PD-L1 expression can be achieved without obtaining a test tissue sample. In some embodiments, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express PD-L1 on the surface of the cells based on an assessment that the proportion of cells in the test tissue sample that express PD-L1 on the cell surface is higher than a predetermined threshold level.

In any of the methods comprising the measurement of PD-L1 expression in a test tissue sample, however, it should be understood that the step comprising the provision of a test tissue sample obtained from a patient is an optional step. It should also be understood that in certain embodiments the “measuring” or “assessing” step to identify, or determine the number or proportion of, cells in the test tissue sample that express PD-L1 on the cell surface is performed by a transformative method of assaying for PD-L1 expression, for example by performing a reverse transcriptase-polymerase chain reaction (RT-PCR) assay or an IHC assay. In certain other embodiments, no transformative step is involved and PD-L1 expression is assessed by, for example, reviewing a report of test results from a laboratory. In certain embodiments, the steps of the methods up to, and including, assessing PD-L1 expression provides an intermediate result that may be provided to a physician or other healthcare provider for use in selecting a suitable candidate for the anti-PD-1 antibody or anti-PD-L1 antibody therapy. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express PD-L1 is assessed by performing an assay to determine the presence of PD-L1 RNA. In further embodiments, the presence of PD-L1 RNA is determined by RT-PCR, in situ hybridization or RNase protection. In other embodiments, the proportion of cells that express PD-L1 is assessed by performing an assay to determine the presence of PD-L1 polypeptide. In further embodiments, the presence of PD-L1 polypeptide is determined by immunohistochemistry (IHC), enzyme-linked immunosorbent assay (ELISA), in vivo imaging, or flow cytometry. In some embodiments, PD-L1 expression is assayed by IHC. In other embodiments of all of these methods, cell surface expression of PD-L1 is assayed using, e.g., IHC or in vivo imaging.

Imaging techniques have provided important tools in cancer research and treatment. Recent developments in molecular imaging systems, including positron emission tomography (PET), single-photon emission computed tomography (SPECT), fluorescence reflectance imaging (FM), fluorescence-mediated tomography (FMT), bioluminescence imaging (BLI), laser-scanning confocal microscopy (LSCM) and multiphoton microscopy (MPM), will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, “In vivo imaging in cancer,” Cold Spring Harb. Perspect. Biol. 2(12):a003848 (2010)). antibody specificity, coupled with the sensitivity and resolution of PET, makes immunoPET imaging particularly attractive for monitoring and assaying expression of antigens in tissue samples (McCabe and Wu, “Positive progress in immunoPET—not just a coincidence,” Cancer Biother. Radiopharm. 25(3):253-61 (2010); Olafsen et al., “ImmunoPET imaging of B-cell lymphoma using 124I-anti-CD20 scFv dimers (diabodies),” Protein Eng. Des. Sel. 23(4):243-9 (2010)). In certain embodiments of any of the present methods, PD-L1 expression is assayed by immunoPET imaging. In certain embodiments of any of the present methods, the proportion of cells in a test tissue sample that express PD-L1 is assessed by performing an assay to determine the presence of PD-L1 polypeptide on the surface of cells in the test tissue sample. In certain embodiments, the test tissue sample is a FFPE tissue sample. In other embodiments, the presence of PD-L1 polypeptide is determined by IHC assay. In further embodiments, the IHC assay is performed using an automated process. In some embodiments, the IHC assay is performed using an anti-PD-L1 mAb to bind to the PD-L1 polypeptide.

In one embodiment of the present methods, an automated IHC method is used to assay the expression of PD-L1 on the surface of cells in FFPE tissue specimens. This disclosure provides methods for detecting the presence of human PD-L1 antigen in a test tissue sample, or quantifying the level of human PD-L1 antigen or the proportion of cells in the sample that express the antigen, which methods comprise contacting the test sample, and a negative control sample, with a mAb that specifically binds to human PD-L1, under conditions that allow for formation of a complex between the antibody or portion thereof and human PD-L1. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In a particular embodiment, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary Ab; incubating with a postprimary blocking agent; incubating with NovoLink Polymer; adding a chromogen substrate and developing; and counterstaining with hematoxylin.

For assessing PD-L1 expression in tumor tissue samples, a pathologist examines the number of membrane PD-L1⁺ tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%. In one embodiment, the threshold number of cells that needs to be PD-L1 positive is at least about 100, at least about 125, at least about 150, at least about 175, or at least about 200 cells. In certain embodiments, the threshold number or cells that needs to be PD-L1 positive is at least about 100 cells.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. The histoscore is calculated as follows:

Histoscore=[(% tumor×1(low intensity))+(% tumor×2(medium intensity))+(% tumor×3(high intensity)]

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 expression in a test tissue sample IHC is to determine the adjusted inflammation score (AIS) score defined as the density of inflammation multiplied by the percent PD-L1 expression by tumor-infiltrating inflammatory cells (Taube et al., “Colocalization of inflammatory response with B7-hl expression in human melanocytic lesions supports an adaptive resistance mechanism of immune escape,” Sci. Transl. Med. 4(127): 127ra37 (2012)).

The present methods can treat a non-squamous NSCLC of any stages. There are at least seven stages used for NSCLC: occult (hidden) stage, Stage 0 (carcinoma in situ), Stage I, Stage II, Stage IIIA, Stage IIIB, and Stage IV. In the occult stage, the cancer cannot be seen by imaging or bronchoscopy. In Stage 0, cancer cells are found in the lining of the airways.

In one embodiment, the present methods treat a Stage I non squamous NSCLC. Stage I NSCLC is divided in Stage IA and IB. In Stage IA, the tumor is in the lung only and is 3 centimeters or smaller. In Stage IB, the cancer has not spread to the lymph nodes and one or more of the following is true: 1) the tumor is larger than 3 centimeters but not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus.

In another embodiment, the methods of the present invention treats a Stage II non squamous NSCLC. Stage II NSCLC is divided into Stage IIA and IIB. In Stage IIA, the cancer has either spread to the lymph nodes or not. If the cancer has spread to the lymph nodes, then the cancer can only have spread to the lymph nodes on the same side of the chest as the tumor, the lymph nodes with cancer or within the lung or near the bronchus. and one or more of the following is true: 1) the tumor is not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIA if the cancer has not spread to the lymph nodes and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. In stage IIB, the cancer has either spread to the lymph nodes or not. If the cancer has spread to the lymph nodes, then the cancer can only have spread to the lymph nodes on the same side of the chest as the tumor, the lymph nodes with cancer are within the lung or near the bronchus and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIB if the cancer has not spread to the lymph nodes and one or more of the following is true: 1) the tumor is larger than 7 centimeters; 2) the cancer has spread to the main bronchus(and is at least 2 centimeters below where the trachea joins the bronchus), the chest wall, the diaphragm, or the nerve that controls the diaphragm; 3) cancer has spread to the membrane around the heart or lining the chest wall; 4) the whole lung has collapsed or developed pneumonitis (inflammation of the lung); or 5) there are one or more separate tumors in the same lobe of the lung.

In other embodiments, any methods of the present invention treats Stage III non squamous NSCLC. Stage IIIA is divided into 3 sections. These 3 sections are based on 1) the size of the tumor; 2) where the tumor is found and 3) which (if any) lymph nodes have cancer. In the first type of Stage IIIA NSCLC, the cancer has spread to the lymph nodes on the same side of the chest as the tumor, and the lymph nodes with the cancer are near the sternum or where the bronchus enters the lung. Additionally: 1) the tumor may be any size; 2) part of the lung (where the trachea joins the bronchus) or the whole lung may have collapsed or developed pneumonitis (inflammation of the lung); 3) there may be one or more separate tumors in the same lobe of the lung; and 4) cancer can have spread to any of the following: a) main bronchus, but not the area where the trachea joins the bronchus, b) chest well, c) diaphragm and the nerve that controls it, d) membrane around the lung or lining the chest wall, e) membrane around the heart. In the second type of Stage IIIA NSCLC, the cancer has spread to the lymph nodes on the same side of the chest as the tumor, and the lymph nodes with the cancer are within the lung or near the bronchus. Additionally: 1) the tumor may be any size; 2) the whole lung may have collapsed or developed pneumonitis (inflammation of the lung); 3) there may be one or more separate tumors in the any of the lobes of the lung with cancer; and 4) cancer can have spread to any of the following: a) main bronchus, but not the area where the trachea joins the bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the third type of Stage IIIA NSCLC, the cancer has not spread to the lymph nodes, the tumor may be any size, and cancer has spread to any one of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi). Stage IIIB is divided into 2 sections depending on 1) the size of the tumor, 2) where the tumor is found, and 3) which lymph nodes have cancer. In the first type of Stage IIIB NSCLC, the cancer has spread to the lymph nodes on the opposite side of the chest as the tumor. Additionally, 1) the tumor may be any size; 2) part of the lung (where the trachea joins the bronchus) or the whole lung may have collapsed or developed pneumonitis (inflammation of the lung); 3) there may be one or more separate tumors in any of the lobs of the lung with cancer; and 4) cancer may have spread to any of the following: a) main bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the second type of Stage IIIB NSCLC, the cancer has spread to lymph nodes on the same side of the chest as the tumor. The lymph nodes with cancer are near the sternum (chest bone) or where the bronchus enters the lung. Additionally, 1) the tumor may be any size; 2) there may be separate tumors in different lobes of the same lung; and 3) cancer has spread to any of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi).

In some embodiments, the methods of the invention treats a Stage IV non squamous NSCLC. In Stage IV NSCLC, the tumor may be any size and the cancer may have spread to the lymph nodes. One or more of the following is true in Stage IV NSCLC: 1) there are one or more tumors in both lungs; 2) cancer is found in the fluid around the lungs or heart; and 3) cancer has spread to other parts of the body, such as the brain, liver, adrenal glands, kidneys or bone.

Anti-PD-1 Antibodies and Anti-PD-L1 Antibodies

Anti-PD-1 antibodies suitable for use in the disclosed methods are antibodies that bind to PD-1 with high specificity and affinity, block the binding of PD-L1, and inhibit the immunosuppressive effect of the PD-1 signaling pathway. In any of the therapeutic methods disclosed herein, an anti-PD-1 or anti-PD-L1 “antibody” includes an antigen-binding portion that binds to the PD-1 or PD-L1 receptor, respectively, and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-L1 antibody or antigen-binding fragment thereof competes for binding with BMS-936559, MPDL3280A, MEDI4736 or MSB0010718C for binding to human PD-L1.

In other embodiments, the anti-PD-1 antibody, or anti-PD-L1 antibody, or antigen-binding portions thereof is a chimeric, humanized or human monoclonal antibody or a portion thereof. In certain embodiments for treating a human subject, the antibody is a humanized antibody In other embodiments for treating a human subject, the antibody is a human antibody antibodies of an IgG1, IgG2, IgG3 or IgG4 isotype can be used.

In certain embodiments, the anti-PD-1 antibody, or anti-PD-L1 antibody, or antigen-binding portions thereof comprises a heavy chain constant region which is of a human IgG1 or IgG4 isotype. In certain other embodiments, the sequence of the IgG4 heavy chain constant region of the anti-PD-1 antibody, or anti-PD-L1 antibody, or antigen-binding portions thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al. In vitro characterization of the anti-PD-1 antibody nivolumab, BMS-936558, and in vivo toxicology in non-human primates, Cancer Imm Res, 2(9):846-56 (2014)). In yet other embodiments, the antibody comprises a light chain constant region which is a human kappa or lambda constant region. In other embodiments, the anti-PD-1 antibody, or anti-PD-L1 antibody, or antigen-binding portions thereof is a mAb or an antigen-binding portion thereof.

HuMAbs that bind specifically to PD-1 with high affinity have been disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and (j) inhibits tumor cell growth in vivo. Anti-PD-1 antibodies usable in the present invention include mAbs that bind specifically to human PD-1 and exhibit at least one, in some embodiments, at least five, of the preceding characteristics. In some embodiments, the anti-PD-1 antibody is nivolumab. In one embodiment, the anti-PD-1 antibody is pembrolizumab.

In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al. In vitro characterization of the anti-PD-1 antibody nivolumab, BMS-936558, and in vivo toxicology in non-human primates, Cancer Imm Res, 2(9):846-56 (2014)). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

In another embodiment, the anti-PD-1 antibody or fragment thereof cross-competes with pembrolizumab. In some embodiments, the anti-PD-1 antibody or fragment thereof binds to the same epitope as pembrolizumab. In certain embodiments, the anti-PD-1 antibody has the same CDRs as pembrolizumab. In another embodiment, the anti-PD-1 antibody is pembrolizumab. Pembrolizumab (also known as KEYTRUDA®, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also http://www.cancer.gov/drugdictionary?cdrid=695789 (last accessed: Dec. 14, 2014). Pembrolizumab has been approved by the FDA for the treatment of relapsed or refractory melanoma.

In other embodiments, the anti-PD-1 antibody or fragment thereof cross-competes with MEDI0608. In still other embodiments, the anti-PD-1 antibody or fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MEDI0608 is described, for example, in U.S. Pat. No. 8,609,089B2 or in http://www.cancer.gov/drugdictionary?cdrid=756047 (last accessed Dec. 14, 2014).

In certain embodiments, the first antibody is an anti-PD-1 antagonist. One example of the anti-PD-1 antagonist is AMP-224, which is a B7-DC Fc fusion protein. AMP-224 is discussed in U.S. Publ. No. 2013/0017199 or in http://www.cancer.gov/publications/dictionaries/cancer-drug?cdrid=700595 (last accessed Jul. 8, 2015).

In other embodiments, the anti-PD-1 antibody or fragment thereof cross-competes with BGB-A317. In some embodiments, the anti-PD-1 antibody or fragment thereof binds the same epitope as BGB-A317. In certain embodiments, the anti-PD-1 antibody has the same CDRs as BGB-A317. In certain embodiments, the anti-PD-1 antibody is BGB-A317, which is a humanized monoclonal antibody. BGB-A317 is described in U.S. Publ. No. 2015/0079109.

Anti-PD-1 antibodies usable in the disclosed methods also include isolated antibodies that bind specifically to human PD-1 and cross-compete for binding to human PD-1 with nivolumab (see, e.g., U.S. Pat. Nos. 8,008,449 and 8,779,105; WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 as, nivolumab are mAbs. For administration to human subjects, these cross-competing antibodies are chimeric antibodies, or humanized or human antibodies. Such chimeric, humanized or human mAbs can be prepared and isolated by methods well known in the art.

Anti-PD-1 antibodies usable in the methods of the disclosed invention also include antigen-binding portions of the above antibodies. It has been amply demonstrated that the antigen-binding function of an antibody can be performed by fragments of a full-length antibody Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab″)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an antibody

Anti-PD-1 antibodies suitable for use in the disclosed compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the antibody is a humanized antibody. In other embodiments, the antibody is a human antibody. Antibodies of an IgG1, IgG2, IgG3 or IgG4 isotype can be used.

In certain embodiments, the anti-PD-1 antibody or antigen-binding portion thereof comprises a heavy chain constant region which is of a human IgG1 or IgG4 isotype. In certain other embodiments, the sequence of the IgG4 heavy chain constant region of the anti-PD-1 antibody or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al. (2014)). In yet other embodiments, the antibody comprises a light chain constant region which is a human kappa or lambda constant region. In other embodiments, the anti-PD-1 antibody or antigen-binding portion thereof is a mAb or an antigen-binding portion thereof. In certain embodiments of any of the therapeutic methods described herein comprising administration of an anti-PD-1 antibody, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), AMP-224, or BGB-A31

In certain embodiments of any of the therapeutic methods described herein comprising administration of an anti-PD-1 antibody, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), or AMP-224.

Anti-PD-1 antibodies usable in the methods of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an antibody.

In certain embodiments, an anti-PD-1 antibody used in the methods can be replaced with another PD-1 or anti-PD-L1 antagonist. For example, because an anti-PD-L1 antibody prevents interaction between PD-1 and PD-L1, thereby exerting similar effects to the signaling pathway of PD-1, an anti-PD-L1 antibody can replace the use of an anti-PD-1 antibody in the methods disclosed herein. Therefore, in one embodiment, the present invention is directed to a method for treating a subject afflicted with a non-squamous NSCLC comprising administering to the subject a therapeutically effective amount an anti-PD-L1 antibody. In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446) (see, e.g., Herbst et al. (2013) J Clin Oncol 31(suppl):3000. Abstract; U.S. Pat. No. 8,217,149), MEDI4736 (also called Durvalumab; Khleif (2013) In: Proceedings from the European Cancer Congress 2013; Sep. 27-Oct. 1, 2013; Amsterdam, The Netherlands. Abstract 802, See U.S. Pat. No. 8,779,108 or US 2014/0356353, filed May 6, 2014), or MSB0010718C (also called Avelumab; See US 2014/0341917).

Because anti-PD-1 and anti-PD-L1 target the same signaling pathway and have been shown in clinical trials to exhibit similar levels of efficacy in a variety of cancers, including RCC (see Brahmer et al. (2012) N Engl J Med 366:2455-65; Topalian et al. (2012a) N Engl J Med 366:2443-54; WO 2013/173223), an anti-PD-L1 antibody can be substituted for the anti-PD-1 antibody in any of the therapeutic methods disclosed herein. In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446) (see, e.g., Herbst et al. (2013) J Clin Oncol 31(suppl):3000. Abstract; U.S. Pat. No. 8,217,149) or MEDI4736 (Khleif (2013) In: Proceedings from the European Cancer Congress 2013; Sep. 27-Oct. 1, 2013; Amsterdam, The Netherlands Abstract 802). In certain embodiments, the antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art.

Combination Therapies with Anti-PD-1 or Anti-PD-L1 Antibodies

In certain embodiments, an anti-PD-1 antibody or anti-PD-L1 antibody is administered in combination with one or more other anti-cancer agents. In certain embodiments, the one or more anti-cancer agents have been administered to the subject prior to the administration of the anti-PD-1 or anti-PD-L1 antibodies or prior to the combination with the anti-PD-1 or anti-PD-L1 antibodies. In certain embodiments, the one or more anti-cancer agents were not effective in treating the cancer. In some embodiments, the other anti-cancer agent is any anti-cancer agent described herein or known in the art. In certain embodiments, the other anti-cancer agent is an anti-CTLA-4 antibody. In one embodiment, the other anti-cancer agent is a chemotherapy or a platinum-based doublet chemotherapy (PT-DC). In certain embodiments, the other anti-cancer agent is an EGFR-targeted tyrosine kinase inhibitor (TKI). In one embodiment, the other anti-cancer agent is an anti-VEGF antibody. In other embodiments, the anti-cancer agent is a platinum agent (e.g., cisplatin, carboplatin), a mitotic inhibitor (e.g., paclitaxel, albumin-bound paclitaxel, docetaxel, taxotere, docecad), a fluorinated Vinca alkaloid (e.g., vinflunine, javlor), vinorelbine, vinblastine, etoposide, or pemetrexed gemcitabin. In one embodiment, the other anti-cancer agent is 5-flurouracil (5-FU). In certain embodiments, the other anti-cancer agent is any other anti-cancer agent known in the art. In some embodiments, two or more additional anti-cancer agents are administered in combination with the anti-PD-1 or anti-PD-L1 antibody. In some embodiments, the PD-1 or PD-L1 antibody is combined with surgical resection and/or radiation therapy. In embodiments, the anti-PD-1 antibody and the additional anti-cancer agent are administered in a fixed dose.

In certain embodiments, the anti-PD-1 antibody or anti-PD-L1 antibody can be combined with another immunotherapy. In certain embodiments, immunotherapy involving blockade of immune checkpoints is administered as a monotherapy. In other embodiments, immunotherapy involving blockade of immune checkpoints is administered in combination with other therapies. Thus, ipilimumab in combination with chemotherapy has exhibited encouraging results in small-cell and non-small-cell lung cancer alike. In some embodiments, NSCLC patients can benefit from the combination of different immunotherapeutic drugs.

Anti-CTLA-4 Antibodies

In certain embodiments, an anti-PD-1 antibody or anti-PD-L1 antibody is combined with an anti-CTLA-4 antibody. Anti-CTLA-4 antibodies useful for the combination can bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

HuMAbs that bind specifically to CTLA-4 with high affinity have been disclosed in U.S. Pat. Nos. 6,984,720 and 7,605,238. Other anti-CTLA-4 mAbs have been described in, for example, U.S. Pat. Nos. 5,977,318, 6,051,227, 6,682,736, and 7,034,121. The anti-CTLA-4 HuMAbs disclosed in U.S. Pat. Nos. 6,984,720 and 7,605,238 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 Abs usable in the present invention include mAbs that bind specifically to human CTLA-4 and exhibit at least one, at least two or, in one embodiment, at least three of the preceding characteristics. An exemplary clinical anti-CTLA-4 antibody is the human mAb 10D1 (now known as ipilimumab and marketed as YERVOY®) as disclosed in U.S. Pat. No. 6,984,720. Ipilimumab is an anti-CTLA-4 antibody for use in the methods disclosed herein. Another anti-CTLA-4 antibody usable in the present methods is tremelimumab.

An exemplary clinical anti-CTLA-4 antibody useful for the combination is the human mAb 10D1 (now known as ipilimumab and marketed as YERVOY®) as disclosed in U.S. Pat. No. 6,984,720. Ipilimumab is an anti-CTLA-4 antibody for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

Another anti-CTLA-4 antibody useful for the present methods is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

Anti-CTLA-4 antibodies usable in the disclosed methods also include isolated antibodies that bind specifically to human PD-1 and cross-compete for binding to human CTLA-4 with ipilimumab or tremelimumab or bind to the same epitope region of human CTLA-4 as ipilimumab or tremelimumab. In certain embodiments, the antibodies that cross-compete for binding to human CTLA-4 with, or bind to the same epitope region of human PD-1 as does ipilimumab or tremelimumab, are antibodies comprising a heavy chain of the human IgG1 isotype. For administration to human subjects, these cross-competing antibodies are chimeric antibodies, or humanized or human antibodies. Usable anti-CTLA-4 antibodies also include antigen-binding portions of the above antibodies such as Fab, F(ab″)₂, Fd or Fv fragments.

Ipilimumab (YERVOY®) is a fully human, IgG1 monoclonal antibody that blocks the binding of CTLA-4 to its B7 ligands, thereby stimulating T cell activation and improving overall survival (OS) in patients with advanced melanoma (Hodi et al. (2010) N Engl J Med 363:711-23). Concurrent therapy with nivolumab and ipilimumab in a Phase 1 clinical trial produced rapid and deep tumor regression in a substantial proportion of patients with advanced melanoma, and was significantly more effective than either antibody alone (Wolchok et al. (2013) N Engl J Med 369(2):122-33; WO 2013/173223). However, it was hitherto not known whether this combination of immunoregulatory antibodies would be similarly effective in other tumor types.

Anti-VEGF Antibody

In other embodiments, an anti-PD-1 antibody or anti-PD-L1 antibody is combined with an anti-VEGF antagonist, e.g., an anti-VEGF antibody. Vascular endothelial growth factor (“VEGF”) is an endothelial cell-specific mitogen and an inducer of angiogenesis. VEGF has a prominent role in angiogenesis and tumor growth and development. In some embodiments of this invention, the anti-PD-1 antibody is administered in combination with an anti-VEGF antagonist. In certain embodiments, the anti-VEGF antagonist is an anti-VEGF antibody, antigen binding molecule or fragment thereof. In certain embodiments, the anti-VEGF antibody is bevacizumab (described in U.S. Pat. No. 7,169,901), or any other VEGF antibody known in the art including ranibizumab (U.S. Pat. No. 7,297,334), VGX-100 (U.S. Pat. No. 7,423,125), r84 (U.S. Pat. No. 8,034,905), aflibercept (U.S. Pat. No. 5,952,199), IMC-18F1 (U.S. Pat. No. 7,972,596), IMC-1C11 (PCT/US2000/02180), and ramucirumab (U.S. Pat. No. 7,498,414).

Chemotherapy and Platinum-Based Chemotherapy

In some embodiments, the anti-PD-1 antibody is administered in combination with any chemotherapy known in the art. In certain embodiments, the chemotherapy is a platinum based-chemotherapy. Platinum-based chemotherapies are coordination complexes of platinum. In some embodiments, the platinum-based chemotherapy is a platinum-doublet chemotherapy. In one embodiment, the chemotherapy is administered at the approved dose for the particular indication. In other embodiments, the chemotherapy is administered at any dose disclosed herein. In some embodiments, the platinum-based chemotherapy is cisplatin, carboplatin, oxaliplatin, satraplatin, picoplatin, Nedaplatin, Triplatin, Lipoplatin, or combinations thereof. In certain embodiments, the platinum-based chemotherapy is any other platinum-based chemotherapy known in the art. In some embodiments, the chemotherapy is the nucleotide analog gemcitabine. In an embodiment, the chemotherapy is a folate antimetabolite. In an embodiment, the folate antimetabolite is pemetrexed. In certain embodiments the chemotherapy is a taxane. In other embodiments, the taxane is paclitaxel. In other embodiments, the chemotherapy is a nucleoside analog. In one embodiment, the nucleoside analog is gemcitabine. In some embodiments, the chemotherapy is any other chemotherapy known in the art. In certain embodiments, at least one, at least two or more chemotherapeutic agents are administered in combination with the anti-PD-1 antibody. In some embodiments, the anti-PD-1 antibody is administered in combination with gemcitabine and cisplatin. In some embodiments, the anti-PD-1 antibody is administered in combination with pemetrexed and cisplatin. In certain embodiments, the anti-PD-1 antibody is administered in combination with gemcitabine and pemetrexed. In one embodiment, the anti-PD-1 antibody is administered in combination with paclitaxel and carboplatin. In an embodiment, an anti-CTLA-4 antibody is additionally administered.

Tyrosine Kinase Inhibitors

In certain embodiments, the anti-PD-1 antibody or an anti-PD-L1 antibody is administered in combination with a tyrosine kinase inhibitor. In certain embodiments, the tyrosine kinase inhibitor is gefitinib, erlotinib, combinations thereof or any other tyrosine kinase inhibitor known in the art. In some embodiments, the tyrosine kinase inhibitor act on the epidermal growth factor receptor (EGFR). In an embodiment, an anti-CTLA-4 antibody is additionally administered.

Pharmaceutical Compositions and Dosages

Therapeutic agents of the present invention can be constituted in a composition, e.g., a pharmaceutical composition containing an antibody and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In one embodiment, the carrier for a composition containing an antibody is suitable for intravenous, intramuscular, subcutaneous, parenteral, spinal or epidermal administration (e.g., by injection or infusion), whereas the carrier for a composition containing a TKI is suitable for non-parenteral, e.g., oral, administration. A pharmaceutical composition of the invention can include one or more pharmaceutically acceptable salts, anti-oxidant, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. For administration of an anti-PD-1 antibody, as a monotherapy or in combination with another anti-cancer agent, the dosage can range from about 0.01 to about 20 mg/kg, about 0.1 to about 10 mg/kg, about 0.1 to about 5 mg/kg, about 1 to about 5 mg/kg, about 2 to about 5 mg/kg, about 7.5 to about 12.5 mg/kg, or about 0.1 to about 30 mg/kg of the subject”s body weight. For example, dosages can be about 0.1, about 0.3, about 1, about 2, about 3, about 5 or about 10 mg/kg body weight, or about 0.3, about 1, about 2, about 3, or about 5 mg/kg body weight. The dosing schedule is typically designed to achieve exposures that result in sustained receptor occupancy (RO) based on typical pharmacokinetic properties of an antibody An exemplary treatment regime entails administration about once per week, once about every 2 weeks, once about every 3 weeks, once about every 4 weeks, once about a month, once about every 3-6 months or longer. In certain embodiments, an anti-PD-1 antibody such as nivolumab is administered to the subject once about every 2 weeks. In other embodiments, the antibody is administered once about every 3 weeks. The dosage and scheduling can change during a course of treatment. For example, a dosing schedule for anti-PD-1 monotherapy can comprise administering the Ab: (i) about every 2 weeks in about 6-week cycles; (ii) about every 4 weeks for about six dosages, then about every three months; (iii) about every 3 weeks; (iv) about 3-about 10 mg/kg once followed by about 1 mg/kg every about 2-3 weeks. Considering that an IgG4 antibody typically has a half-life of 2-3 weeks, a dosage regimen for an anti-PD-1 antibody of the invention comprises at least about 0.3-at least about 10 mg/kg body weight, at least about 1-at least about 5 mg/kg body weight, or at least about 1-at least about 3 mg/kg body weight via intravenous administration, with the antibody being given every about 14-21 days in up to about 6-week or about 12-week cycles until complete response or confirmed progressive disease. In certain embodiments, an anti-PD-1 monotherapy is administered at 3 mg/kg every 2 weeks until progressive disease or unacceptable toxicity. In some embodiments, the antibody treatment, or any combination treatment disclosed herein, is continued for at least about 1 month, at least about 3 months, at least about 6 months, at least about 9 months, at least about 1 year, at least about 18 months, at least about 24 months, at least about 3 years, at least about 5 years, or at least about 10 years.

When used in combinations with other cancer agents, the dosage of an anti-PD-1 antibody can be lowered compared to the monotherapy dose. Dosages of nivolumab that are lower than the typical 3 mg/kg, but not less than 0.001 mg/kg, are subtherapeutic dosages. The subtherapeutic doses of an anti-PD-1 antibody used in the methods herein are higher than 0.001 mg/kg and lower than 3 mg/kg. In some embodiments, a subtherapeutic dose is about 0.001 mg/kg-about 1 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.5 mg/kg, or at least about 1.0 mg/kg body weight. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%). In some embodiments, 0.3 mg/kg dosing can allow for sufficient exposure to lead to maximal biologic activity. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al. (2010) J Clin Oncol 28:3167-75). Thus, 0.3 mg/kg dosing can allow for sufficient exposure to lead to maximal biologic activity.

Although higher nivolumab monotherapy dosing up to about 10 mg/kg every two weeks has been achieved without reaching the maximum tolerated does (MTD), the significant toxicities reported in other trials of checkpoint inhibitors plus anti-angiogenic therapy (see, e.g., Johnson et al. (2013) Cancer Immunol Res 1:373-77; Rini et al. (2011) Cancer 117:758-67) support the selection of a nivolumab dose lower than 10 mg/kg.

In certain embodiments, the dose of an anti-PD-1 antibody (or an anti-PD-L1 antibody) is a fixed dose in a pharmaceutical composition. In other embodiments, the method of the present invention can be used with a flat dose (a dose given to a patient irrespective of the body weight of the patient). For example, a flat dose of a nivolumab can be about 240 mg. For example, a flat dose of pembrolizumab can be about 200 mg. In embodiments, the anti-PD-1 antibody or antigen-binding portion thereof is administered at a dose of about 240 mg. In embodiments, the anti-PD-1 antibody or antigen-binding portion thereof is administered at a dose of about 360 mg. In embodiments, the anti-PD-1 antibody or antigen-binding portion thereof is administered at a dose of about 480 mg. In one embodiment, 360 mg of the anti-PD-1 antibody or antigen binding fragment is administered once every 3 weeks. In another embodiment, 480 mg of the anti-PD-1 antibody or antigen binding fragment is administered once every 4 weeks.

Ipilimumab (YERVOY®) is approved for the treatment of melanoma at 3 mg/kg given intravenously once every 3 weeks for 4 doses. Thus, in some embodiments, about 3 mg/kg is the highest dosage of ipilimumab used in combination with the anti-PD-1 antibody though, in certain embodiments, an anti-CTLA-4 antibody such as ipilimumab can be dosed within the range of about about 0.3 to about 10 mg/kg, about 0.5 to about 10 mg/kg, about 0.5 to about 5 mg/kg, or about 1 to about 5 mg/kg. body weight about every two or three weeks when combined with nivolumab. In other embodiments, ipilimumab is administered on a different dosage schedule from nivolumab. In some embodiments, ipilimumab is administered about every week, about every two weeks, about every three weeks, about every 4 weeks, about every five weeks, about every six weeks, about every seven weeks, about every eight weeks, about every nine weeks, about every ten weeks, about every eleven weeks, about every twelve weeks or about every fifteen weeks. Dosages of ipilimumab that are lower than the typical 3 mg/kg every 3 weeks, but not less than 0.001 mg/kg, are subtherapeutic dosages. The subtherapeutic doses of an anti-CTLA-4 antibody used in the methods herein are higher than 0.001 mg/kg and lower than 3 mg/kg. In some embodiments, a subtherapeutic dose is about 0.001 mg/kg-about 1 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.5 mg/kg, or at least about 1.0 mg/kg body weight. It has been shown that combination dosing of nivolumab at 3 mg/kg and ipilimumab at 3 mg/kg exceeded the MTD in a melanoma population, whereas a combination of nivolumab at 1 mg/kg plus ipilimumab at 3 mg/kg or nivolumab at 3 mg/kg plus ipilimumab at 1 mg/kg was found to be tolerable in melanoma patients (Wolchok et al. (2013) N Engl J Med 369(2):122-33). Accordingly, although nivolumab is tolerated up to 10 mg/kg given intravenously every 2 weeks, in certain embodiments doses of the anti-PD-1 antibody do not exceed about 3 mg/kg when combined with ipilimumab. In certain embodiments, based on risk-benefit and PK-PD assessments, the dosage used comprises a combination of nivolumab at about 1 mg/kg plus ipilimumab at about 3 mg/kg, nivolumab at about 3 mg/kg plus ipilimumab at about 1 mg/kg, or nivolumab at about 3 mg/kg plus ipilimumab at about 3 mg/kg is used, each administered at a dosing frequency of once about every 2-4 weeks, in certain embodiments, once about every 2 weeks or once about every 3 weeks. In certain other embodiments, nivolumab is administered at a dosage of about 0.1, about 0.3, about 1, about 2, about 3 or about 5 mg/kg in combination with ipilimumab administered at a dosage of about 0.1, about 0.3, about 1, about 2, about 3 or about 5 mg/kg, once about every 2 weeks, once about every 3 weeks, or once about every 4 weeks.

In certain embodiments, the combination of an anti-PD-1 antibody and an anti-CTLA-4 antibody is administered intravenously to the subject in an induction phase about every 2 or 3 weeks for 1, 2, 3 or 4 administrations. In certain embodiments, the combination of nivolumab and ipilimumab is administered intravenously in the induction phase about every 2 weeks or about every 3 weeks for about 4 administrations. The induction phase is followed by a maintenance phase during which only the anti-PD-1 antibody is administered to the subject at a dosage of about 0.1, about 0.3, about 1, about 2, about 3, about 5 or about 10 mg/kg about every two or three weeks for as long as the treatment proves efficacious or until unmanageable toxicity or disease progression occurs. In certain embodiments, nivolumab is administered during the maintenance phase at a dose of about 3 mg/kg body about every 2 weeks.

In certain embodiments, the anti-PD-1 antibody and the anti-CTLA-4 antibody is formulated as a single composition, wherein the dose of the anti-PD-1 antibody and the dose of the anti-CTLA-4 antibody are combined in a fixed dose at a ratio of 1:50, 1:40, 1:30, 1:20, 1:10. 1:5, 1:3, 1:1, 3:1, 5:1, 10:1, 20:1, 30:1, 40:1, or 50:1. In other embodiments, the dose of the anti-CTLA-4 antibody is a fixed dose. In certain embodiments, the dose of the anti-CTLA-4 antibody is a flat dose, which is given to a patient irrespective of the body weight. In a specific embodiment, the flat dose of the anti-CTLA-4 antibody is about 80 mg.

For combination of nivolumab with other anti-cancer agents, these agents are administered at their approved dosages. Treatment is continued as long as clinical benefit is observed or until unacceptable toxicity or disease progression occurs. Nevertheless, in certain embodiments, the dosages of these anti-cancer agents administered are significantly lower than the approved dosage, i.e., a subtherapeutic dosage, of the agent is administered in combination with the anti-PD-1 antibody The anti-PD-1 antibody can be administered at the dosage that has been shown to produce the highest efficacy as monotherapy in clinical trials, e.g., about 3 mg/kg of nivolumab administered once about every three weeks (Topalian et al. (2012a) N Engl J Med 366:2443-54; Topalian et al. (2012b) Curr Opin Immunol 24:207-12), or at a significantly lower dose, i.e., at a subtherapeutic dose. In certain embodiments, the anti-PD-1 antibody is administered at about 3 mg/kg once about every two weeks.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, or until the patient shows partial or complete amelioration of symptoms of disease. Thereafter, the patient can be administered a prophylactic regime.

Actual dosage levels of the active ingredient or ingredients in the pharmaceutical compositions of the present invention can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

Kits

Also within the scope of the present invention are kits comprising an anti-PD-1 antibody and, optionally, another anti-cancer agent for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a lung cancer, the kit comprising: (a) a dosage ranging from at least about 0.1 to at least about 10 mg/kg body weight of an anti-cancer agent which is an antibody or an antigen-binding portion thereof that specifically binds to the PD-1 receptor and inhibits PD-1 activity; and, optionally, (b) a dosage of another anti-cancer agent which is any anti-cancer agent described herein, including: (i) a platinum-based doublet chemotherapy; (ii) an EGFR-targeted tyrosine kinase inhibitor; (iii) bevacizumab; or (iv) a dosage ranging from at least about 0.1 to at least about 10 mg/kg body weight of an antibody or an antigen-binding portion thereof that specifically binds to and inhibits CTLA-4; and (c) instructions for using the anti-PD-1 antibody and, optionally, the other anti-cancer agent in any of the therapy methods disclosed herein. In certain embodiments, the anti-PD-1, the anti-CTLA-4 antibody and/or the TKI can be co-packaged in unit dosage form. In certain embodiments for treating human patients, the kit comprises an anti-human PD-1 antibody disclosed herein, e.g., nivolumab or pembrolizumab. In other embodiments, the kit comprises an anti-human CTLA-4 antibody disclosed herein, e.g., ipilimumab or tremelimumab.

The present invention is further illustrated by the following example, which should not be construed as further limiting. The contents of all references cited throughout this application are expressly incorporated herein by reference.

### Example 1

Treatment of Advanced Non-Squamous NSCLC with Nivolumab or Docetaxel

Study Design

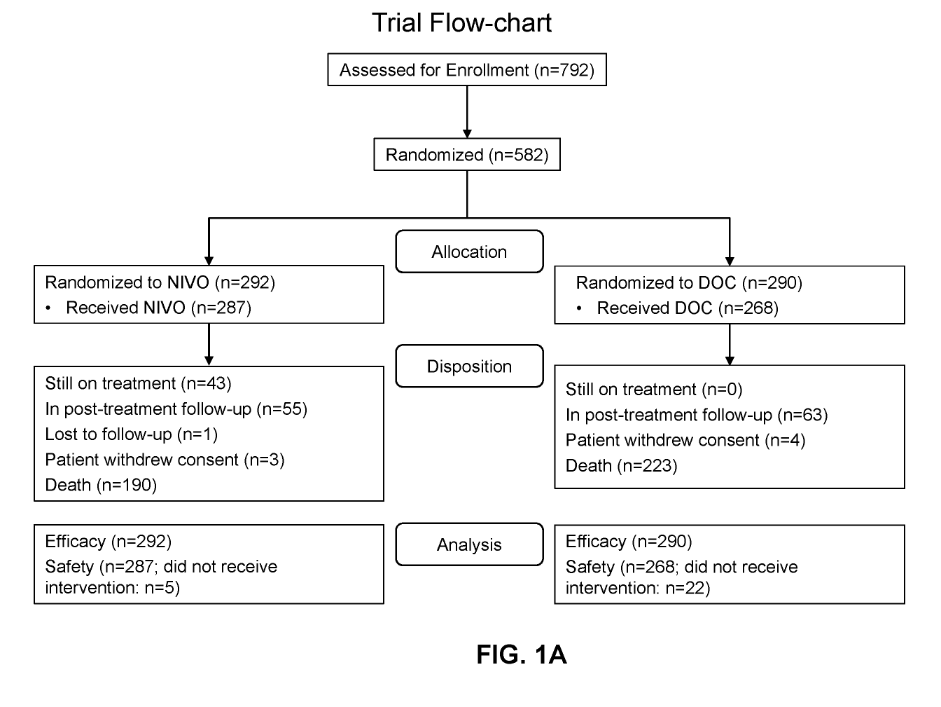

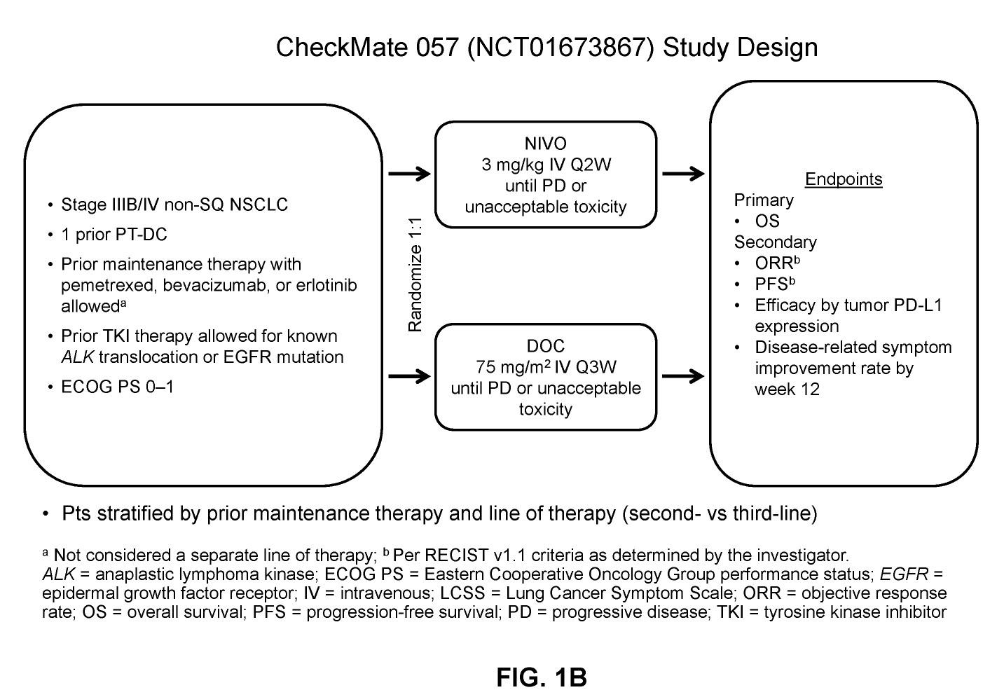

This data is based on a randomized, global, phase III study that evaluated the efficacy and safety of nivolumab (nivo) versus docetaxel (doc) in patients with advanced non-squamous (non-SQ) NSCLC after failure of PT-DC (March 2015 database lock). 582 patients were randomized to receive either nivolumab or docetaxel. 292 were randomized to receive nivolumab, and of these, 287 actually received nivolumab. 290 were randomized to receive docetaxel, and of these, 268 actually received docetaxel. Patients were treated with nivolumab at 3 mg/kg IV Q2W until they experienced a progressive disease or unacceptable toxicity. Patients were treated with docetaxel at 75 mg/m² IV Q3W until progressive disease or unacceptable toxicity occurred. The primary end point of the study was overall survival (OS). The secondary endpoints of the study were overall response rate (ORR) (Per RECIST v1.1 criteria as determined by the investigator), progression free survival (PFS) (Per RECIST v1.1 criteria as determined by the investigator), efficacy by tumor PD-L1 expression, and disease-related symptom improvement rate by week 12. An overview of the trial can be seen in FIGS. 1A and 1B.

All patients had either stage IIIB or stage IV non-squamous NSCLC and 1 prior PT-DC treatment. Prior maintenance therapy with pemetrexed, bevacizumab or erlotinib was allowed (and was not considered a separate line of therapy). Prior TKI therapy was allowed for a known ALK translocation or EGFR mutation. The ECOG PS was 0-1. Patients were stratified by prior maintenance therapy and line of therapy (second-vs third-line)

In this example, ALK means anaplastic lymphoma kinase; ECOG PS means Eastern Cooperative Oncology Group performance status; EGFR means epidermal growth factor receptor; IV means intravenous; LCSS means Lung Cancer Symptom Scale; ORR means objective response rate; OS=overall survival; PFS=progression-free survival; PD=progressive disease; and TKI means tyrosine kinase inhibitor.

The baseline demographics of the patients and a summary of the prior therapy is seen below in Table 1.

TABLE 1
Baseline Characteristics.

| | Nivo | Doc |
|---|---|---|
| | N = 292 | N = 290 |
| Median age, years (range) | 61 (37, 84) | 64 (21, 85) |
| ≥75 years, % | 6.8 | 7.9 |
| Gender, % | | |
| Male | 52 | 58 |
| ECOG PS,¹ % | | |
| 0 | 29 | 33 |
| 1 | 71 | 67 |
| EGFR-positive mutation status % | 15 | 13 |
| ALK-positive translocation status, % | 4.5 | 2.8 |
| CNS metastasis, % | 12 | 12 |
| Prior maintenance therapy, % | 42 | 38 |
| Number of prior systematic regimens,²³ | | |
| 1 | 88 | 89 |
| 2 | 12 | 11 |
| Time from completion of most recent prior | 82 | 82 |
| systemic regimen, % <6 months | | |
| Best response to most recent prior systemic | | |
| regimen, % | | |
| CR or PR | 25 | 23 |
| SD | 35 | 33 |
| PD | 38 | 40 |

CNS = central nervous system;
CR = complete response;
PR = partial response;
SD = stable disease
¹1 patient in the docetaxel arm had ECOG PS = 3
²Derived as number of lines of prior therapy received for advanced, metastatic or recurrent disease
³1 pt had 1 prior regimen in neo-adjuvant setting.

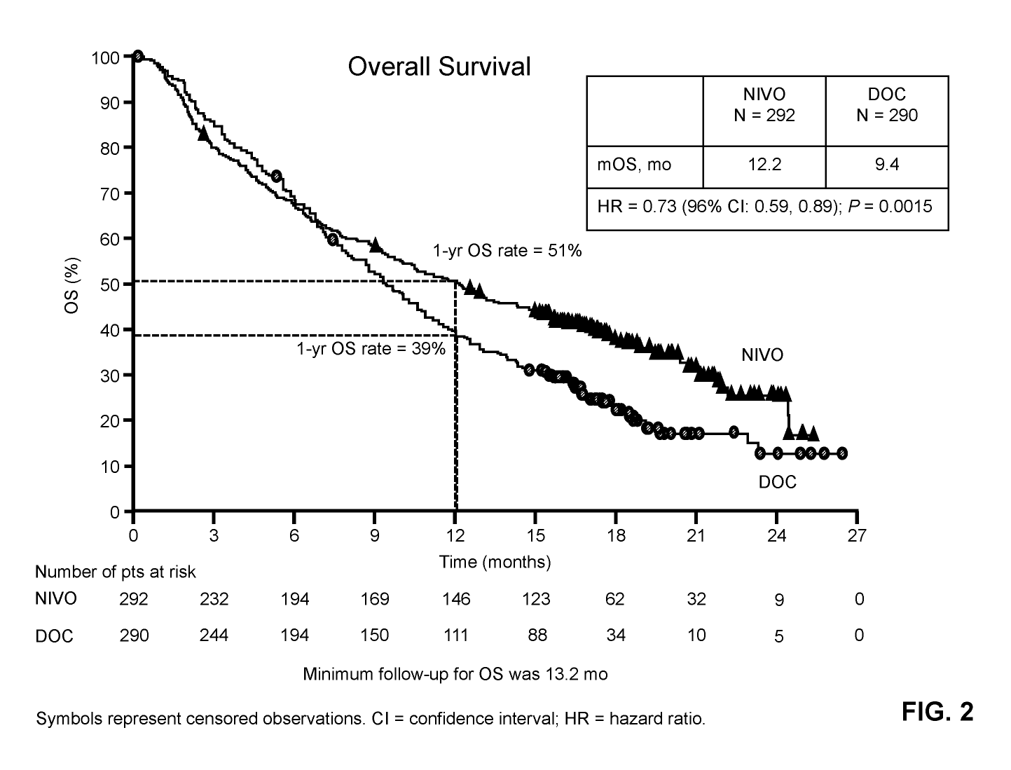

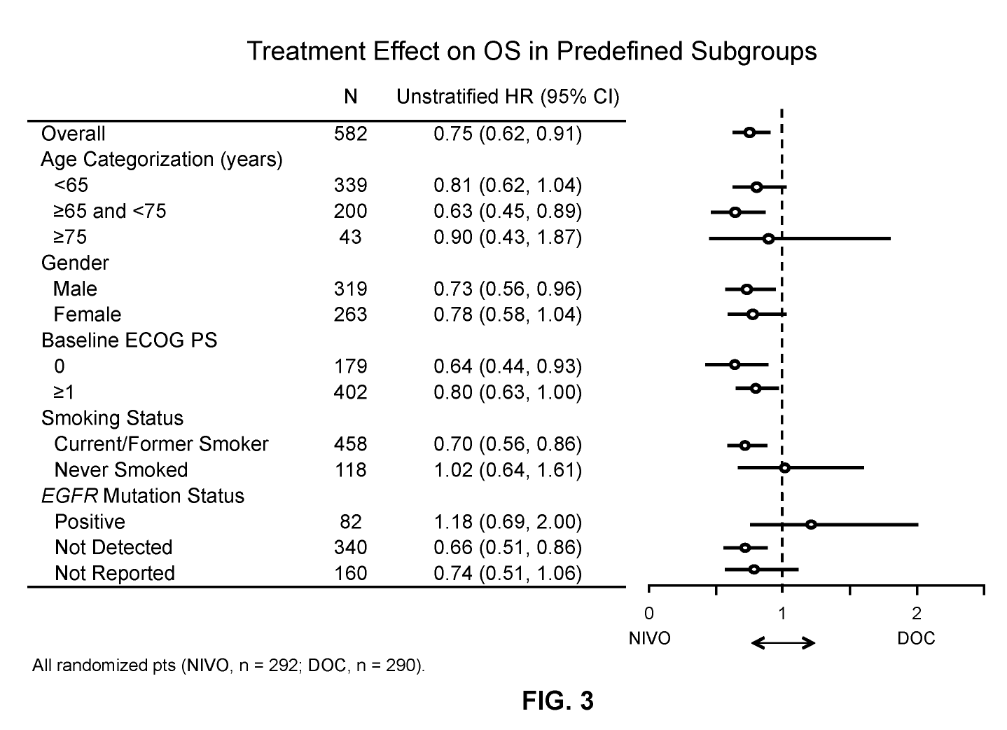

The overall survival of patients receiving nivolumab and patients receiving docetaxel can be seen in FIG. 2. The minimum follow-up for overall survival (OS) was 13.2 months. At one year, the OS for patients receiving nivolumab was 51%, while the OS for patients receiving docetaxel was 39%. The treatment effect on the OS in predefined subgroups can be seen in FIG. 3. The tumor response to the treatment can be seen in Table 2.

TABLE 2
Tumor Response

| | Nivo | | Doc | |
|---|---|---|---|---|
| | N = 292 | | N = 290 | |
| ORR, n (%) [95% CI] | 59 (19) [15, 24] | | 36 (12) [8.8, 17) | |
| Estimated odds ratio (95% CI) | 1.7 (1.1, 2.6) | | | |
| P-value | 0.0246 | | | |
| Best overall response, n (%) | | | | |
| CR | 4 | (1.4) | 1 | (0.3) |
| PR | 52 | (18) | 35 | (12) |
| SD | 74 | (25) | 122 | (42) |
| PD | 129 | (44) | 85 | (29) |
| Unable to determine | 33 | (11) | 47 | (16) |
| Median time to response,⁴ | 2.1 | (1.2, 8.6) | 2.6 | (1.4, 6.3) |
| mos (range) | | | | |
| Median DOR,^{a} mos (range) | 17.2 | (1.8, 22.6+) | 5.6 | (1.2+, 15.2+) |
| Ongoing response,⁵ n (%) | 29 | (52) | 5 | (14) |

Symbol + indicates a censored value.
⁴Values are for all responders (NIVO, n = 56; DOC, n = 36).
⁵Ongoing response at last tumor assesssment before censoring.

71 pts were treated beyond RECIST v1.1-defined progression. A non-conventional benefit was observed in 16 pts (not included in best overall response).

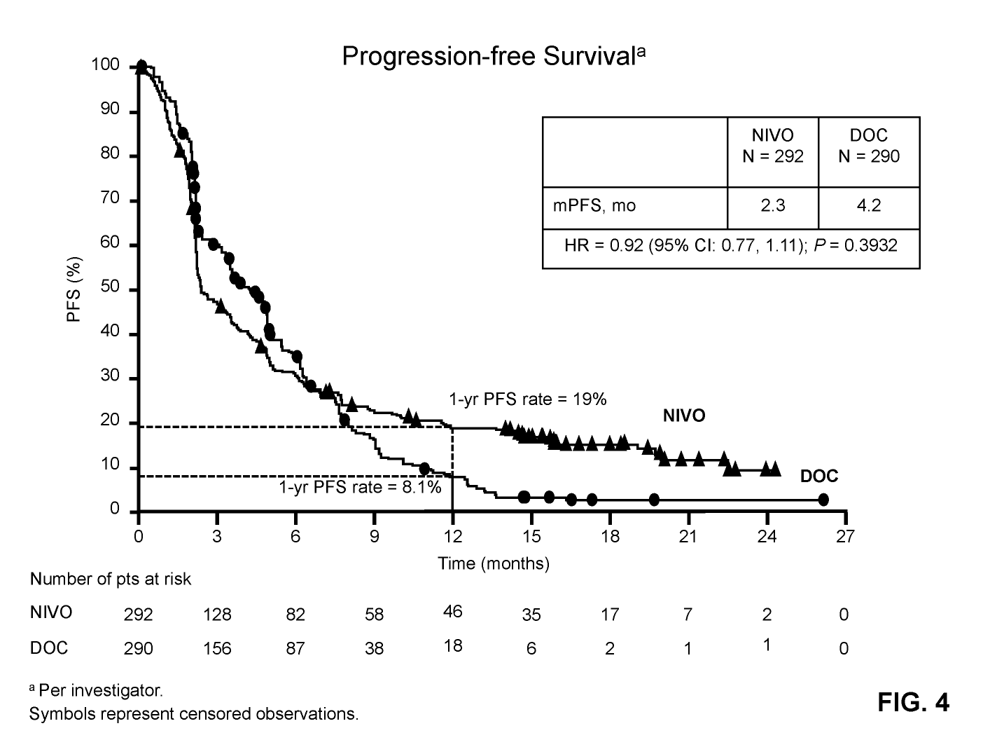

The progression-free survival (PFS) of patients receiving nivolumab and patients receiving docetaxel can be seen in FIG. 4. The 1-year PFS rate for patients receiving nivolumab was 19%, while the 1-year PFS rate for patients receiving docetaxel was 8.1%.

The PD-L1 expression was measured for the patients. The DAKO PD-L1 Assay Analytical Validation and Clinical Sample Testing is described in Table 3.

TABLE 3
DAKO PD-L1 Assay Analytical Validation
and Clinical Sample Testing.

| | Results |
|---|---|
| Robustness | Lot-to-lot comparisons yielded <5% variability for low |
| | expressing samples |
| Sensitivity | Assessment of PD-L1 expression demonstrated staining |
| | across a range of 0-100 positive tumor cells and 0-3 |
| | staining intensity |
| | 74 cases of each SQ NSCLC (73% positive at ≥1%, |
| | 43% positive at ≥5%) |
| | 112 cases of non-SQ NSCLC (72% positive at ≥1%, |
| | 48% positive at ≥5%) |
| Specificity | Staining results of PD-L1 in assessed normal tissues |
| | are consistent with published literature |
| | 30 tissues and 3 normal specimens per tissue type |
| Reproducibility | All variables tested met pre-determined acceptance |
| | criteria of >85% agreement for SQ and non-SQ samples |
| | at expression levels of 1%, 5% and 10% |
| | Assessed reagent, operator, pathologist variability |
| | over multiple days |

PD-L1 expression was measured in pre-treatment (archival or recent) tumor biopsies using the Dako automated IHC assay (rabbit anti-human PD-L1 antibody, clone 28-8)

A summary of the baseline tumor PD-L1 expression is seen in Table 4.

TABLE 4
Baseline Tumor PD-L1 expression.

| | PD-L1 expression level | | | | | | |
|---|---|---|---|---|---|---|---|
| | 1% | | 5% | | 10% | | |
| | Positive | Negative | Positive | Negative | Positive | Negative | Not |
| | (≥1%) | (<1%) | (≥5%) | (<5%) | (≥10%) | (<10%) | quantifiable |
| Nivo, % | 42 | 37 | 33 | 47 | 29 | 50 | 21 |
| Doc, % | 42 | 35 | 30 | 48 | 27 | 50 | 23 |

78% (455/582) of randomized patients had quantifiable PD-L1 expression

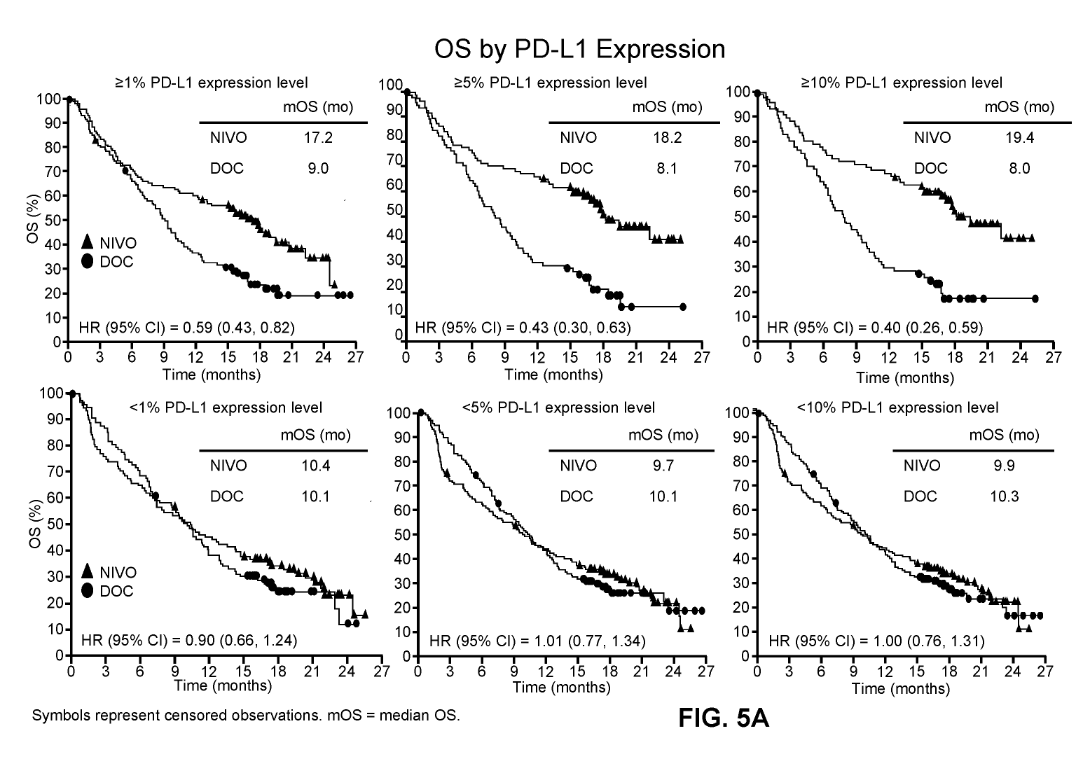

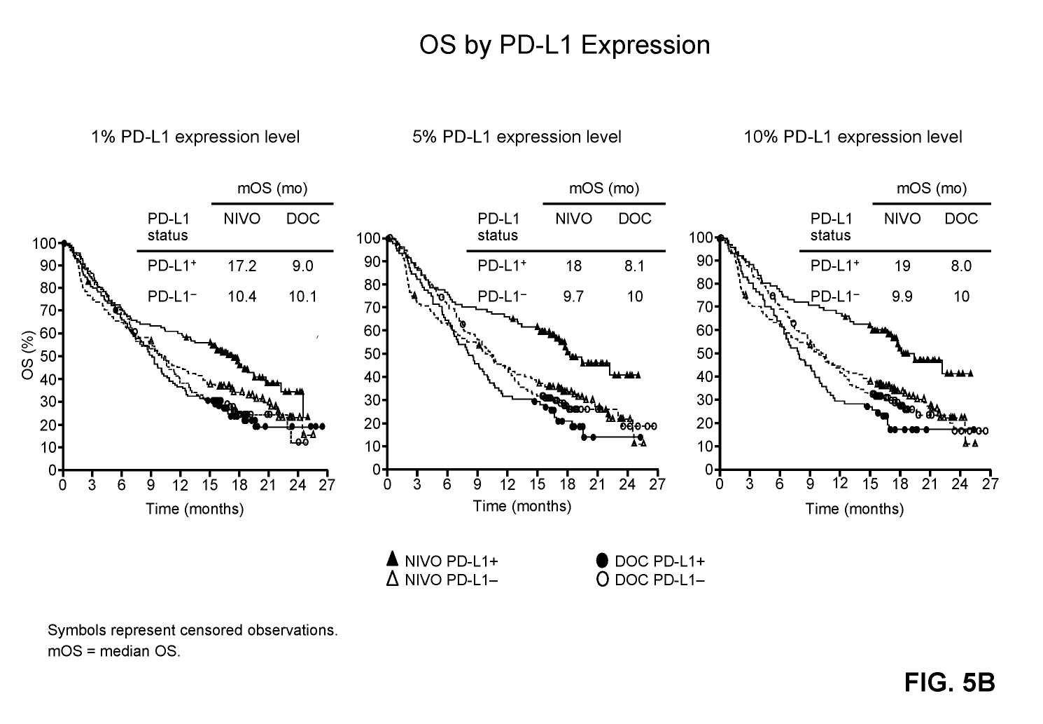

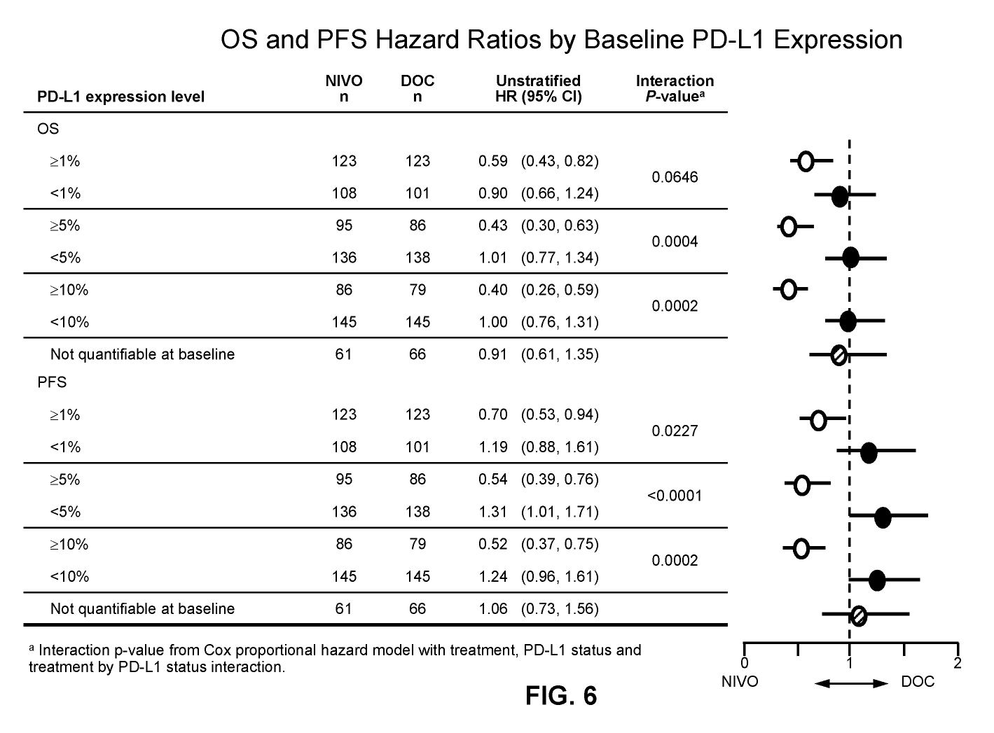

The overall survival by PD-L1 expression can be seen in FIGS. 5(A) and (B). The overall survival and progression-free survival hazard ratios by baseline PD-L1 expression can be seen in FIG. 6. The overall response rate by PD-L1 express can be seen in Table 5.

TABLE 5
ORR by PD-L1 Expression.

| PD-L1 expression level | | | | | | |
|---|---|---|---|---|---|---|
| 1% | | 5% | | 10% | | |
| Positive | Negative | Positive | Negative | Positive | Negative | Not |
| (≥1%) | (<1%) | (≥5%) | (<5%) | (≥10%) | (<10%) | quantifiable |

Nivo

| | | | | | | | |
|---|---|---|---|---|---|---|---|
| ORR,⁶ % | 31 | 9.3 | 36 | 10 | 37 | 11 | 13 |
| Median | 16.0 | 18.3 | 16.0 | 18.3 | 16.0 | 18.3 | 7.3 |
| DOR, mos | | | | | | | |
| (95% CI) | (8.4, NE) | (4.2, NE) | (8.4, NE) | (5.5, NE) | (6.9, NE) | (7.5, NE) | (2.2, NE) |
| n | 38 | 10 | 34 | 14 | 32 | 16 | 8 |

DOC

| | | | | | | | |
|---|---|---|---|---|---|---|---|
| ORR,⁶ % | 12 | 15 | 13 | 14 | 13 | 14 | 9.1 |
| Median | 5.6 | 5.6 | 5.6 | 5.6 | 5.6 | 5.6 | 6.6 |
| DOR, mos | | | | | | | |
| (95% CI) | (3.0, 5.7) | (4.2, 9.9) | (3.0, 7.0) | (4.2, 7.1) | (1.6, 6.2) | (4.2, 7.1) | (2.8, 14.2) |
| n | 15 | 15 | 11 | 19 | 10 | 20 | 6 |

NE = not evaluable
⁶CR + PR as per RECIST v1.1 criteria. Confirmation of response required (investigator assessment)
NE = not evaluable

Table 6 shows the exposure and safety summary of the clinical trial. Table 7 shows treatment-related select adverse events.

TABLE 6
Exposure and Safety Summary.

| | Nivo | Doc |
|---|---|---|
| | N = 287 | N = 268 |
| Median number of | 6 (1, 52) | 4 (1, 23) |
| doses received | | |
| (range) | | |
| Relative dose | | |
| intensity % | | |
| ≥90% | 83 | 66 |
| 70 to <90 | 15 | 26 |
| <70% | 2.8 | 5.2 |
| Pts continuing | 43 | 0 |
| treatment, % | | |

| | Any | Grade | Any | Grade |
|---|---|---|---|---|
| | Grade | 3-4⁷ | Grade | 3-4⁷ |
| Treatment-related | 69 | 10 | 88 | 54 |
| AEs % | | | | |
| Treatment-related | 3.7 | 5.2 | 20 | 18 |
| SAEs, % | | | | |
| Treatment-related | 4.9 | 3.8 | 15 | 6.7 |
| AEs leading to | | | | |
| discontinuation, % | | | | |

| | | |
|---|---|---|
| Treatment-related | 0⁸ | 0.4⁹ |
| deaths | | |

AE = adverse event; SAE = serious adverse event.
⁷No grade 5 events were reported at DBL.
⁸1 death attributed to NIVO (encephalitis); association to NIVO changed after DBL
⁹1 death attributed to DOC-related drug toxicity; grade 4 febrile neutropenia.

TABLE 7
Treatment-related Select AEs

| | |
|---|---|
| Nivo | Doc |
| N = 287 | N = 268 |

| | Any | Grade | Any | Grade |
|---|---|---|---|---|
| | Grade | 3-4¹⁰ | Grade | 3-4¹⁰ |
| Endocrine | | | | |
| Hypothyroidism, % | 6.6 | 0 | 0 | 0 |
| Gastrointestinal, % | | | | |
| Diarrhea | 7.7 | 0.7 | 23 | 1.1 |
| Hepatic, % | | | | |
| ALT increased | 3.1 | 0 | 1.5 | 0.4 |
| AST increased | 3.1 | 0.3 | 0.7 | 0 |
| Pulmonary, % | | | | |
| Pneumonitis | 2.8 | 1.0 | 0.4 | 0.4 |
| Skin, % | | | | |
| Rash | 9.4 | 0.3 | 3.0 | 0 |
| Pruritus | 8.4 | 0 | 1.5 | 0 |
| Erythema | 1.4 | 0 | 4.1 | 0 |
| Hypersensitivity/ | | | | |
| infusion reaction, % | | | | |
| Infusion-related | 2.8 | 0 | 3.0 | 0.4 |
| reaction | | | | |

Includes events reported in ≥2.5% of pts.
ALT = alanine aminotransferase; AST = aspartate aminotransferase.
¹⁰No grade 5 events were reported at DBL

### CONCLUSIONS

Nivolumab is the first PD-1 inhibitor to significantly improve overall survival vs current standard-of-care docetaxel in previously treated patients with advanced non-SQ NSCLC. A 27% reduction in risk of death (HR=0.73; P=0.0015) was seen with nivolumab in comparison to docetaxel. The 1-yr OS with nivolumab was 51% vs 39% with docetaxel. The median OS was 12.2 months with nivolumab vs. 9.4 months with docetaxel.

Nivolumab also showed an improved response profile vs docetaxel. The ORR with nivolumab was 19% vs 12% with docetaxel (P=0.0246). The median DOR with nivolumab was 17.2 months vs 5.6 months with docetaxel.

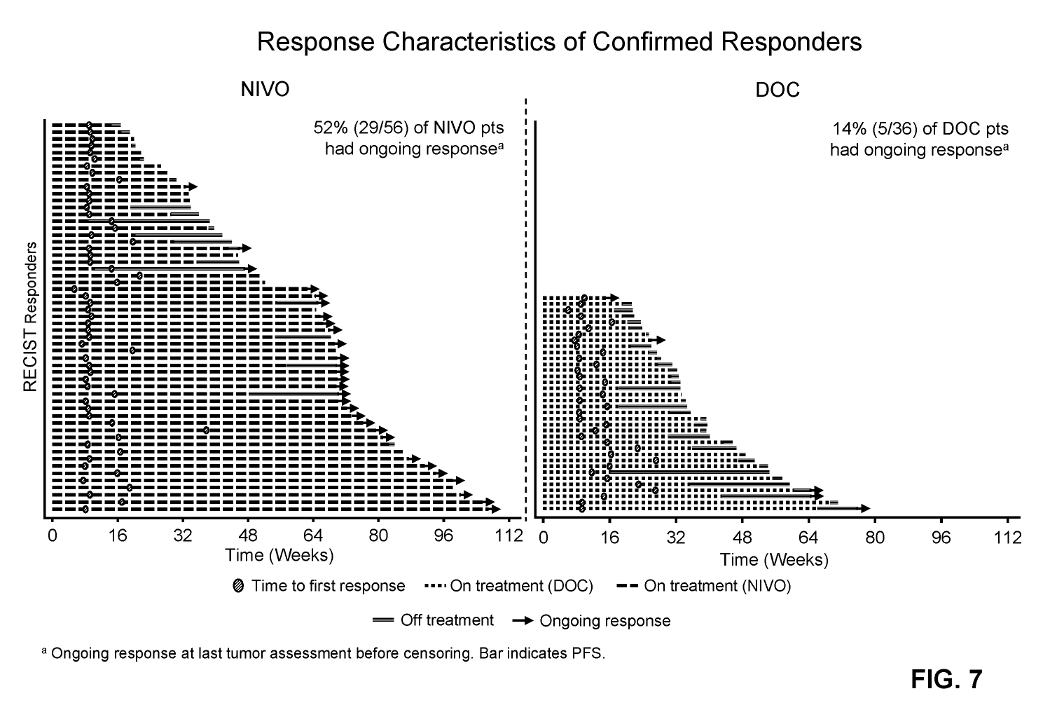

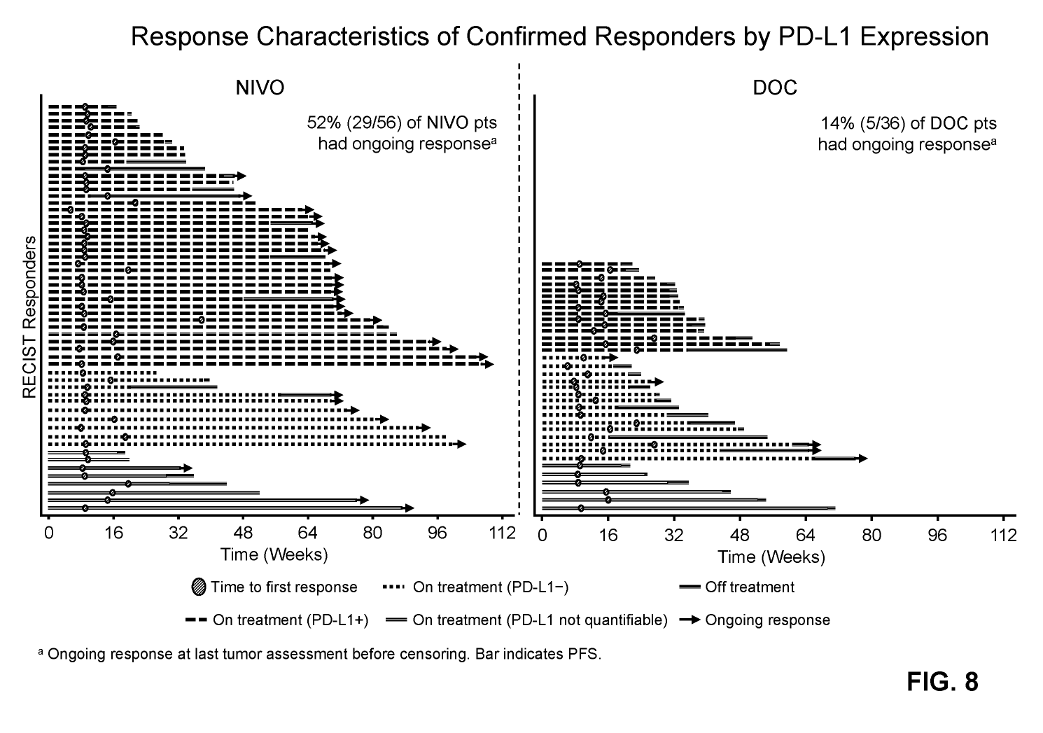

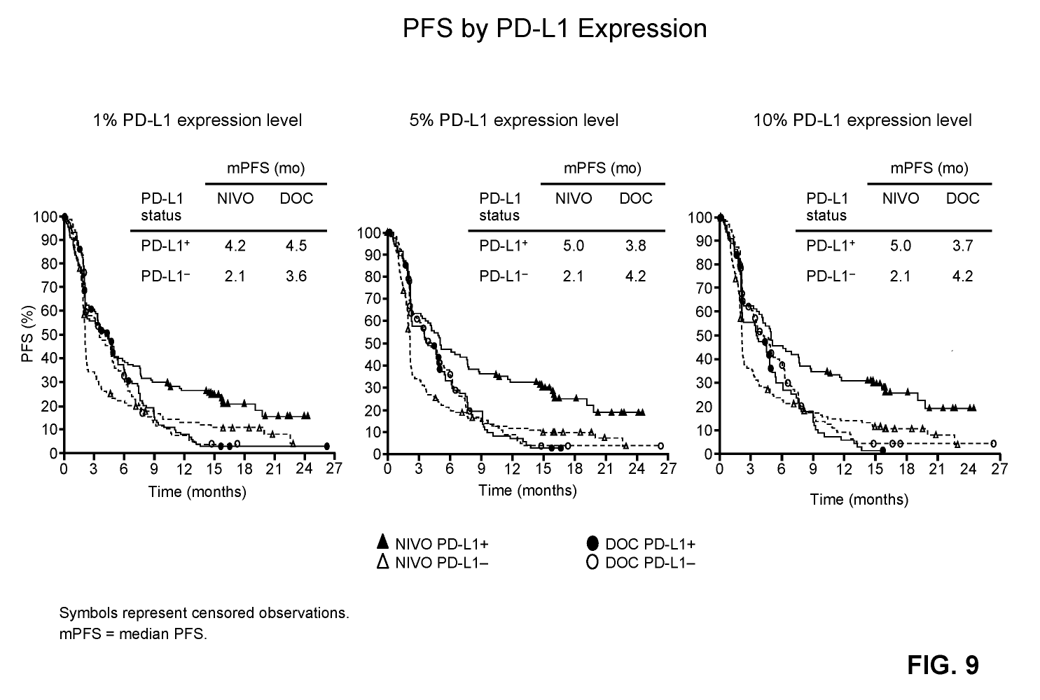

FIG. 7 shows the response characteristics of confirmed responders, while FIG. 8 shows the response characteristics of confirmed responders by PD-L1 expression. FIG. 9 shows the progression-free survival by PD-L1 expression.

Among PD-L1⁺ patients, the median OS nearly doubled when treating with nivolumab vs docetaxel. The mOS (nivolumab vs docetaxel) at 1%, 5%, and 10% expression levels were 17.2 vs. 9.0 months, 18.2 vs. 8.1 months, and 19.4 vs. 8.0 months, respectively. PD-L1 expression appears to be predictive starting at the lowest expression level (1%). There were no OS differences in PD-L1⁻ pts across all expression levels

Additionally, the safety profile of nivolumab was favorable vs docetaxel and consistent with prior studies.

The present application claims benefit to U.S. Provisional Application No. 62/167,674, filed May 28, 2016; which is incorporated herein by reference in its entirety.

What is claimed is:

1. A method for treating a subject afflicted with a tumor derived from a non-squamous non-small cell lung cancer (NSCLC) comprising administering to the subject a therapeutically effective amount of:
an antibody or an antigen-binding portion thereof that binds specifically to a Programmed Death-1 receptor (PD-1) or Programmed Death-Ligand 1 (PD-L1) and inhibits PD-1 activity (“anti-PD-1 antibody”) or PD-L1 activity (“anti-PD-L1 antibody”), respectively,
wherein the tumor has a PD-L1 expression of at least about 1%.

2. A method of treating a subject afflicted with a tumor derived from a non-squamous NSCLC comprising:
(i) measuring a PD-L1 expression on the tumor, wherein the tumor has a PD-L1 expression of at least about 1% and
(ii) administering to the subject a therapeutically effective amount of an anti-PD-1 antibody or antigen-binding portion thereof or an anti-PD-L1 antibody or antigen-binding portion thereof.

3. A method for identifying a subject afflicted with a tumor derived from a non-squamous NSCLC who is suitable for an anti-PD-1 antibody or anti-PD-L1 antibody therapy comprising measuring a PD-L1 expression on the tumor, wherein the tumor has a PD-L1 expression of at least about 1% and wherein the subject is administered a therapeutically effective amount of an anti-PD-1 antibody or antigen-binding portion thereof or an anti-PD-L1 antibody or antigen-binding portion thereof.

4. A method for identifying a subject afflicted with a tumor derived from a non-squamous NSCLC who is suitable for an anti-PD-1 antibody or anti-PD-L1 antibody therapy comprising:
(i) measuring a PD-L1 expression on the tumor, wherein the tumor has a PD-L1 expression of at least about 1% and
(ii) administering to the subject a therapeutically effective amount of an anti-PD-1 antibody or antigen-binding portion thereof or an anti-PD-L1 antibody or antigen-binding portion thereof.

5. The method of any one of claims 1-4, wherein the PD-L1 expression of the tumor is at least about 5%.

6. The method of any one of claims 1-4, wherein the PD-L1 expression of the tumor is at least about 10%.

7. The method of any one of claims 1-6, wherein the anti-PD-1 antibody or antigen-binding portion thereof cross-competes with nivolumab for binding to human PD-1.

8. The method of any one of claims 1-7, wherein the anti-PD-1 antibody or antigen-binding portion thereof is a chimeric, humanized or human monoclonal antibody or a portion thereof.

9. The method of any one of claims 1-8, wherein the anti-PD-1 antibody or antigen-binding portion thereof comprises a heavy chain constant region which is of a human IgG1 or IgG4 isotype.

10. The method of any one of claims 1-9, wherein the anti-PD-1 antibody is nivolumab.

11. The method of any one of claims 1-9, wherein the anti-PD-1 antibody is pembrolizumab.

12. The method of any one of claim 1-6, wherein the anti-PD-L1 antibody or antigen-binding fragment thereof competes for binding with BMS-936559, MPDL3280A, MEDI4736 or MSB0010718C for binding to human PD-L1.

13. The method of any one of claims 1-6 and 12, wherein the anti-PD-L1 antibody or antigen-binding portion thereof is a chimeric, humanized or human monoclonal antibody or a portion thereof.

14. The method of any one of claims 1-6 and 12-13, wherein the anti-PD-L1 antibody or antigen-binding portion thereof comprises a heavy chain constant region which is of a human IgG1 or IgG4 isotype.

15. The method of any one of claims 1-6 and 12-14, wherein the anti-PD-L1 antibody is BMS-936559.

16. The method of any one of claims 1-6 and 12-14, wherein the anti-PD-L1 antibody is MPDL3280A.

17. The method of any one of claims 1-6 and 12-14, wherein the anti-PD-L1 antibody is MEDI4736.

18. The method of any one of claims 1-6 and 12-14, wherein the anti-PD-L1 antibody is MSB0010718C.

19. The method of any one of claims 1-18, wherein the anti-PD-1 antibody or antigen-binding portion thereof or the anti-PD-L1 antibody or antigen-binding portion thereof is administered at a dose ranging from at least about 0.1 to at least about 10.0 mg/kg body weight once every about 2, about 3 or about 4 weeks.

20. The method of claim 19, wherein the anti-PD-1 antibody or antigen-binding portion thereof or the anti-PD-L1 antibody or antigen-binding portion thereof is administered at a dose of at least about 3 mg/kg body weight once about every 2 weeks.

21. The method of any of claims 1-20, wherein the anti-PD-1 antibody or antigen-binding portion or the anti-PD-L1 antibody or antigen-binding portion thereof is administered for as long as clinical benefit is observed or until unmanageable toxicity or disease progression occurs.

22. The method of any one of claims 1-21, wherein the tumor has a PD-L1 expression that is at least about 5%.

23. The method of any one of claims 1-22, wherein the tumor has a PD-L1 expression that is at least about 10%.

24. The method of any one of claims 1-24, wherein the subject has had prior treatment with platinum-based doublet chemotherapy (PT-DC).

25. The method of any one of claims 1-25, wherein the subject exhibits an overall survival of at least about 10 months, at least about 11 months, at least about 12 months, at least about 13 months, at least about 14 months at least about 15 months, at least about 16 months, at least about 17 months, at least about 18 months, at least about 19 months, at least about 20 months, at least about 21 months, at least about 22 months, at least about 23 months, at least about 2 years, at least about 3 years, at least about 4 years, or at least about 5 years after the administration.

26. The method of claim 25, wherein the tumor has a PD-L1 expression of at least about 1% and the subject exhibits the overall survival of at least about 17 months.

27. The method of claim 25, wherein the tumor has a PD-L1 expression of at least about 5% and the subject exhibits the overall survival of at least about 18 months.

28. The method of claim 25, wherein the tumor has a PD-L1 expression of at least about 10% and the subject exhibits the overall survival of at least about 19 months.

29. The method of any one of claims 1-28, wherein the subject has an overall response rate of at least about 30%, 35%, 36%, 37%, 39%, 40%, 45%, or 50% after the administration compared to the response rate after administration of Docetaxel.

30. The method of claim 29, wherein the tumor has a PD-L1 expression of at least about 1% and the subject exhibits an overall response rate of at least about 31%.

31. The method of claim 29, wherein the tumor has a PD-L1 expression of at least about 5% and the subject exhibits an overall response rate of at least about 35%.

32. The method of claim 29, wherein the tumor has a PD-L1 expression of at least about 10% and the subject exhibits an overall response rate of at least about 37%.

33. The method of any one of claims 1-32, wherein the subject exhibits a median duration of response of at least about 16 months.

34. The method of any one of claims 1-33, wherein the anti-PD-1 antibody or portion thereof or the anti-PD-L1 antibody or portion thereof is formulated for intravenous administration.

35. The method of any one of claims 1-34, wherein the anti-PD-1 antibody or antigen-binding portion thereof or the anti-PD-L1 antibody or antigen-binding portion thereof is administered at a subtherapeutic dose.

36. The method of any one of claims 1 to 35, which further comprising administering one or more additional anti-cancer agent.

37. The method of claim 36, wherein the anti-cancer agent is selected from the group consisting of an antibody or antigen-binding portion thereof that binds specifically to a CTLA-4 and inhibits CTLA-4 activity, a chemotherapy, a platinum-based doublet chemotherapy, a tyrosine kinase inhibitor, or an anti-VEGF inhibitor.

38. The method of claim 36, wherein the anti-cancer agent is an antibody or antigen-binding portion thereof that binds specifically to a CTLA-4 and inhibits CTLA-4 activity.

39. The method of any one of claims 1 to 38, wherein the PD-L1 expression is measured by automated immunohistochemistry (IHC).

40. A kit for treating a subject afflicted with a tumor derived from a non-squamous NSCLC, the kit comprising:
(a) an anti-PD-1 antibody or antigen-binding portion thereof or an anti-PD-L1 antibody or antigen-binding portion thereof;
(b) instructions for determining the PD-L1 expression of the tumor and, if the tumor expresses PD-L1, administering the anti-PD-1 antibody or antigen-binding portion thereof or an anti-PD-L1 antibody or antigen-binding portion thereof to the subject in the methods of claims 1-39.

41. The kit of claim 40, further comprising an agent to determine the PD-L1 expression of the tumor.

42. The kit of claim 41, wherein the PD-L1 expression is measured by an anti-PD-L1 antibody or antigen-binding portion thereof.
